SEQUENCE LISTING (10 sequences)

SEQ ID NO: 1 - protein, 257 aa, Artificial Sequence
  Synthetic Construct
DYKDDDDKLSYELTQPPSVSVSPGQTARITCSGDALPKKYAYWYQQKSGQAPVLVIYEDSKRPSGIPERFSGSSSGTMATLTISGAQVEDEADYYCYSTDSSGNYRVFGGGTKLTVLGEGKSSGSGSESKASEVQLVQSGADVKKPGASLTISCQASGYTFTDYWISWVRQKPGKGLEWMGRIDPSDSYTNYGPSFQGHVTISADRSTATAYLQWRSLEASDTAMYYCVRSSMVTSGGTIPEYYQHWGPGTLVTVSS

SEQ ID NO: 2 - protein, 20 aa, Artificial Sequence
  Synthetic Construct
GELHDLRGQVAKLEAALGEA

SEQ ID NO: 3 - protein, 20 aa, Artificial Sequence
  Synthetic Construct
RIDSLSAQLSQLQKQLAAKE

SEQ ID NO: 4 - protein, 22 aa, Artificial Sequence
  Synthetic Construct
DEYQELLDIKLALDMEIHAYRK

SEQ ID NO: 5 - protein, 20 aa, Artificial Sequence
  Synthetic Construct
SGSPVSASTLARAGFLYTGE

SEQ ID NO: 6 - protein, 20 aa, Artificial Sequence
  Synthetic Construct
THADYLLRTGQVVDISDTIY

SEQ ID NO: 7 - protein, 19 aa, Artificial Sequence
  Synthetic Construct
AEAVDKCPMCYTVITFKQK

SEQ ID NO: 8 - protein, 20 aa, Artificial Sequence
  Synthetic Construct
GFFIIRASQSSPGDFSISVR

SEQ ID NO: 9 - protein, 20 aa, Artificial Sequence
  Synthetic Construct
SLNKLVDYYRTNSISRQKQI

SEQ ID NO: 10 - protein, 20 aa, Artificial Sequence
  Synthetic Construct
TDPVQLQAAGRVRWARALYD

(19) United States
(12) Patent Application Publication
(10) Pub. No.: US 2018/0010118 A1
(43) Pub. Date: Jan. 11, 2018

(54) METHODS OF IDENTIFYING AND VALIDATING AFFINITY REAGENTS

(71) Applicant: AxioMx, Inc., Branford, CT (US)

(72) Inventors: Michael WEINER, Guilford, CT (US); Valeria BUSYGINA, North Haven, CT (US)

(21) Appl. No.: 15/547,680

(22) PCT Filed: Feb. 12, 2016
(86) PCT No.: PCT/US2016/017795

§ 371 Date: Jul. 31, 2017

Related U.S. Application Data
(60) Provisional application No. 62/118,322, filed on Feb. 19, 2015.

(51) Int. Cl. C12N 15/10; C07K 16/00

(52) U.S. Cl. CPC C12N 15/1055; C12N 15/1037; C07K 16/00; C07K 2317/567; C07K 2317/622

(57) ABSTRACT

The invention features methods of identifying and validating affinity reagents, such as antibodies. The methods of the invention generally involve screening an antibody library by, for example, phage display on bacteria (e.g., E. coli) to identify particular antibody clones capable of binding a desired target polypeptide. Clones identified in this way can then be validated using yeast 2-hybrid. In some instances, antibodies identified by their capacity to binding a partial antigen can be validated by their capacity to bind to the full-length antigen. Validated clones can be further screened by additional rounds of phage display and/or yeast 2-hybrid. Between each round, additional variants of particular antibody clones can be generated and screened to identify variants that demonstrate higher binding affinity to the target of interest.

### SEQUENCE LISTING

The instant application contains a Sequence Listing, which has been submitted electronically in ASCII format and is hereby incorporated by reference in its entirety. Said ASCII copy, created on Feb. 12, 2016, is named 50881_011WO2_Sequence_Listing_2_12_16_ST25.txt and is 5,150 bytes in size.

### FIELD OF THE INVENTION

The present invention relates to identification and validation of affinity reagents as capable of binding to target polypeptides.

### BACKGROUND

The most commonly used method for generating antibodies is through the immunization of animals. However, this method is generally low-throughput, expensive, time-consuming, and the antibodies generated are not always renewable. In addition, there is no intermediate validation step to ensure that such antibodies isolated against partial-antigens will effectively bind to full-length antigen. The alternative is the generation of recombinant antibodies (rAbs), currently accomplished through several means: (i) mouse hybridomas and (ii) recombinant display technologies. Mouse hybridoma methods are expensive and can result in a heterogenic collection of affinity reagents. Furthermore, hybridoma-based methods can often take several months to generate rAbs, and do not result in DNA sequence information without further manipulation. Recombinant display technologies, which are animal-free, can be automated and can generate useful rAbs in just weeks. rAbs generated through display technologies are renewable through over-expression in the appropriate heterologous host, and are easily stored and transferred as DNA. However, the cost of current display technologies is high and their throughput is low. Thus, there exists a need in the art for the development of efficient, cost-effective, and broadly available methods for generating renewable affinity reagents, such as recombinant antibodies.

### SUMMARY OF THE INVENTION

The invention features methods of identifying and validating affinity reagents, such as antibodies (e.g., IgGs, scFvs, Fabs, and other antibody types as known in the art). The methods of the invention generally involve screening an antibody library by, for example, phage display on bacteria (e.g., E. coli) to identify particular antibody clones capable of binding a desired target polypeptide. Clones identified in this way can then be validated using a two-hybrid system (e.g., yeast 2-hybrid). In some instances, the antibodies can be identified by their capacity to bind to a partial antigen (partial Ag). In certain instances, antibodies identified by their capacity to bind a partial antigen can be validated by their capacity to bind to the full-length antigen (FL-Ag). Validated clones can be further screened by additional rounds of phage display and/or yeast 2-hybrid. Between each round, variants of particular clones can be further modified, for example, by the AXM cloning methods described herein, to generate additional variants of the clone that can be screened to identify variants that demonstrate higher binding affinity to the target of interest.

In a first aspect, the invention features a method of identifying and validating a binding moiety as capable of binding to a target polypeptide. The method involves:
(a) providing a plurality of viruses, each virus including:
   a nucleic acid encoding a binding moiety, in which the binding moiety is displayed on the surface of the virus;
(b) incubating the plurality of the viruses with the target polypeptide or a peptide fragment thereof;
(c) examining whether the binding moieties displayed by the viruses bind to the target polypeptide, or to the peptide fragment thereof;
(d) expressing, for each of the binding moieties identified as capable of binding to the target polypeptide, in a cell:
   (i) a first fusion protein including a particular binding moiety identified as capable of binding to the target polypeptide and a first reporter moiety, and
   (ii) a second fusion protein including the target polypeptide, or a peptide fragment thereof, and a second reporter moiety,
   in which binding of the particular binding moiety to the target polypeptide, or to the peptide fragment thereof, results in expression in the cell of a detectable gene, in which expression of the detectable gene is under the control of the first reporter moiety and the second reporter moiety; and
(e) determining if the detectable gene is expressed by each of the cells, thereby validating the binding moieties identified as capable of binding to the target polypeptide as capable of binding to the target polypeptide.

In some embodiments of the first aspect, the method further involves, after the examining step and prior to the expressing step, sequencing the nucleic acid encoding each of the binding moieties identified as binding to the target polypeptide or peptide fragment thereof. Methods of sequencing such nucleic acids may include, for example, any sequencing method known in the art (e.g., deep sequencing and Sanger sequencing).

In some embodiments of the first aspect, a plurality of binding moieties are identified in the examining step and the sequencing step as capable of binding to the target polypeptide, and the method further includes expressing each of the identified binding moieties in a distinct cell according to step (d) and determining if the detectable gene is expressed by each of the distinct cells according to step (e). In certain embodiments, the method further includes generating a plurality of variants of at least one of the validated binding moieties and repeating steps (a)-(e) using the plurality of variants as the binding moieties of step (a). In particular embodiments, steps (a)-(e) are repeated at least two times (e.g., 2, 3, 4, 5, 6, 7, 8, 9, or 10 times). In one embodiment, steps (a)-(e) are repeated once.

In a second aspect, the invention features a method of validating a binding interaction between a binding moiety and a target polypeptide. The method involves:

expressing, in a cell:
(a) a first fusion protein including a binding moiety and a first reporter moiety, and
(b) a second fusion protein including a target polypeptide, or a peptide fragment thereof, and a second reporter moiety;
the binding moiety having been identified as capable of binding to the target polypeptide by:
   (i) expressing a nucleic acid encoding the binding moiety on a virus, in which the binding moiety is displayed on the surface of the virus,
   (ii) incubating the virus with the target polypeptide, or the peptide fragment thereof, and
   (iii) examining whether the binding moiety displayed by the virus binds to the target polypeptide, or to the peptide fragment thereof;
in which binding of the binding moiety to the target polypeptide, or to the peptide fragment thereof, in the cell results in the cell expressing a detectable gene, in which expression of the detectable gene is under the control of the first reporter moiety and the second reporter moiety; and

determining if the detectable gene is expressed by the cell, thereby validating the binding moiety as capable of binding to the target polypeptide.

In some embodiments of the second aspect, the method further includes repeating the expressing step and the determining step with one or more additional binding moieties having been identified as capable of binding to the target polypeptide according to steps (i)-(iii).

In some embodiments of any of the above aspects, the binding moiety is an antibody or antibody fragment. In certain embodiments, the binding moiety is a single-chain variable fragment (scFv). In particular embodiments, the scFv includes an antibody framework including an amino acid sequence sharing at least 90% sequence identity (e.g., 90%, 95%, 96%, 97%, 98%, 99%, or 100% sequence identity) with SEQ ID NO: 1.

In some embodiments of any of the above aspects, the incubation step includes incubating the virus with a peptide fragment of the target polypeptide. In certain embodiments, the peptide fragment of the target polypeptide is less than about 40 amino acids in length (e.g., about 5, 6, 7, 8, 9, 10, 11, 12, 13, 14, 15, 16, 17, 18, 19, 20, 25, 30, 35, or 40 amino acids in length). In other embodiments, the peptide fragment of the target polypeptide is greater than about 40 amino acids in length (e.g., about 5, 6, 7, 8, 9, 10, 11, 12, 13, 14, 15, 16, 17, 18, 19, 20, 25, 30, 35, or 40 amino acids in length). In further embodiments, the peptide fragment of the target polypeptide is about 30-100 amino acids in length (e.g., about 30, 35, 40, 45, 50, 55, 60, 65, 70, 75, 80, 85, 90, 95, or 100 amino acids in length). In one embodiment, the peptide fragment is synthetic.

In some embodiments of any of the above aspects, the second fusion protein includes the full length amino acid sequence of the target polypeptide.

In some embodiments of any of the above aspects, the target polypeptide is post-translationally modified. In certain embodiments, the target polypeptide is phosphorylated. In particular embodiments, the target polypeptide is phosphorylated by co-expressing a modifying enzyme (e.g., a kinase) in the cell. In further embodiments, the target polypeptide is site-specifically modified by use of a suppressing cell (e.g., a suppressing yeast or bacterial cell). In one embodiment, the target polypeptide is post-translationally modified in vivo, for example, by use of the pSer system in E. coli or by incorporation of iodo-tyrosine in a two hybrid system (e.g., a yeast two-hybrid system or a bacterial two-hybrid system).

In some embodiments of any of the above aspects, the binding moiety is post-translationally modified (e.g., phosphorylated). In certain embodiments, a suppressing host cell capable of site-specifically modifying the binding moiety is used.

In some embodiments of any of the above aspects, the target polypeptide is a soluble protein. In certain embodiments, the target polypeptide is an intracellular protein (e.g., a cytoplasmic protein, a nuclear protein, a mitochondrial protein, a lysosomal protein, or a chloroplast protein). In one embodiment, the target polypeptide is a cytoplasmic protein. In alternate embodiments, the target polypeptide is a secreted protein. In various embodiments, the target polypeptide is a precursor protein (e.g., a precursor to a secreted protein).

In some embodiments of any of the above aspects, the target polypeptide is a soluble domain of a protein. In certain embodiments, the target polypeptide is a soluble domain of a membrane-bound protein (e.g., an intracellular domain or an extracellular domain).

In some embodiments of any of the above aspects, the first reporter moiety is a transcription factor activation domain and the second reporter moiety is a DNA binding domain. In alternate embodiments, the first reporter moiety is a DNA binding domain and the second reporter moiety is a transcription factor activation domain. In particular embodiments, the transcription factor activation domain is a B42 activation domain. In specific embodiments, the DNA binding domain is a GAL4 DNA binding domain. In one embodiment, the transcription factor activation domain is a B42 activation domain and the DNA binding domain is a GAL4 DNA binding domain.

In some embodiments of any of the above aspects, the cell is a yeast cell, mammalian cell, bacterial cell, insect cell, or plant cell. In some embodiments, the cell is a yeast cell. In certain embodiments, the yeast cell is Saccharomyces cerevisiae or Schizosaccharomyces pombe. In one embodiment, the yeast cell is Saccharomyces cerevisiae. In alternate embodiments, the cell is a mammalian cell (e.g., a human or mouse cell). In further embodiments, the cell is a bacterial cell (e.g., an E. coli cell).

In some embodiments of any of the above aspects, the virus is bacteriophage M13.

In some embodiments of any of the above aspects, the examining step includes performing an enzyme-linked immunosorbent assay (ELISA), immunoprecipitation, Western blot, flow cytometry, or mass spectrometry.

In some embodiments of any of the above aspects, each of the viruses originates from a bi-functional vector including a gene encoding a particular binding moiety, in which: if the bi-functional vector is present in a first cell, the bi-functional vector acts as a template for expression by the first cell of a first fusion protein including the particular binding moiety and a viral protein; and if the bi-functional vector is present in a second cell, the bi-functional vector acts as a template for expression by the second cell of a second fusion protein including the particular binding moiety and a reporter moiety.

In certain embodiments, the first cell is a bacterial cell. In various embodiments, the second cell is a yeast cell and the reporter moiety is a transcription factor activation domain or a DNA binding domain. In particular embodiments, the transcription factor activation domain is a B42 activation domain. In specific embodiments, the DNA binding domain is a GAL4 DNA binding domain. In one embodiment, the transcription factor activation domain is a B42 activation domain and the DNA binding domain is a GAL4 DNA binding domain.

In certain embodiments, the bi-functional vector includes a suppressible stop codon located between the viral protein and the binding moiety. In particular embodiments, the suppressible stop codon is an amber stop codon.

In certain embodiments, the viral protein is a gp3 protein. In one embodiment, the bi-functional vector is a pCH103 vector (such as a pCH103 vector described herein, or a variant thereof).

In some embodiments of any of the above aspects, the expressing step and the determining step are performed in liquid media.

In some embodiments of any of the above aspects, the cell lacks a selectable marker prior to the expressing step, and the expressing step further includes expressing the selectable marker in the cell. In one embodiment, the selectable marker is URA3.

In some embodiments of any of the above aspects, the nucleic acids encoding the binding moieties in the plurality of viruses are generated by:
(i) providing a template DNA molecule including a binding moiety sequence,
(ii) providing a pair of oligonucleotides, in which the oligonucleotides hybridize to opposite strands of the binding moiety sequence, in which one of the oligonucleotides is protected, the other oligonucleotide is non-protected, and the oligonucleotides flank the binding moiety sequence;
(iii) performing an amplification reaction on the template DNA molecule using the oligonucleotides, thereby generating a population of dsDNA variants of the binding moiety sequence;
(iv) incubating the population of dsDNA variants with an enzyme capable of selectively degrading the non-protected strand over the protected strand of the dsDNA variants, thereby producing a population of ssDNA variants of the binding moiety sequence;
(v) hybridizing the population of ssDNA variants to ssDNA intermediaries, in which the ssDNA intermediaries include a sequence substantially identical to the binding moiety sequence or a fragment thereof, generating heteroduplex DNA; and
(vi) transforming the heteroduplex DNA into host cells, thereby generating a plurality of variants of the binding moiety sequence.

In certain embodiments, the template DNA molecule further includes viral nucleic acid sequences. In various embodiments, the method further includes cloning the variants of the binding moiety sequence into a viral vector. In particular embodiments, the nonrecombinant copies of the binding moiety sequence include a predetermined restriction site, and recombinant copies of the binding moiety sequence do not include the predetermined restriction site. In specific embodiments, the host cells express a restriction enzyme (e.g., Eco29kl) that recognizes and cleaves the predetermined restriction site. In one embodiment, the transformation step further includes incubating the host cells under conditions in which the restriction enzyme can cleave nucleic acids having the predetermined restriction site. In any of the above embodiments, the host cells may be bacteria, for example, E. coli (e.g., AXE688 E. coli).

In certain embodiments, the template DNA molecule is a viral vector.

In certain embodiments, the template DNA molecule is a bi-functional vector, in which: if the bi-functional vector is present in a first cell, the bi-functional vector acts as a template for expression by the first cell of a first fusion protein including the binding moiety sequence and a viral protein; and if the bi-functional vector is present in a second cell, the bi-functional vector acts as a template for expression by the second cell of a second fusion protein including the binding moiety sequence and a reporter moiety.

In one embodiment, the bi-functional vector is a pCH103 vector (e.g., a pCH103 vector as described herein, or a variant thereof).

In certain embodiments, the enzyme capable of selectively degrading the non-protected strand over the protected strand of the dsDNA variants is a T7 exonuclease.

In some embodiments of any of the above aspects, the method further includes, after the determining step, immunoprecipitating the target polypeptide from a transiently-transfected cell. In certain embodiments, the target polypeptide to be immunoprecipitated is tagged with an epitope tag. In particular embodiments, the epitope tag is FLAG, HA, Myc, His, V5, GFP, YFP, GST, or MBP. In specific embodiments, the transiently-transfected cell is a mammalian cell (e.g., a human cell or a mouse cell).

In a third aspect, the invention features an antibody framework including an amino acid sequence sharing at least 90% sequence identity (e.g., 90%, 95%, 96%, 97%, 98%, 99%, or 100% sequence identity) with SEQ ID NO: 1. The invention further features a nucleic acid encoding the antibody framework including an amino acid sequence sharing at least 90% sequence identity (e.g., 90%, 95%, 96%, 97%, 98%, 99%, or 100% sequence identity) with SEQ ID NO: 1.

### Definitions

As used herein, “partial antigen” or “partial Ag” means any polypeptide fragment of a full-length protein useful as an antigen, e.g., for the production of antibodies according to the methods of the invention. A “partial antigen” or “proxy antigen” may refer to, for example, any of the following: synthetic peptides, protein fragments, or recombinant protein fusions that do not include the full-length antigen.

By “full-length antigen” or “FL-Ag” is meant the full-length polypeptide of an antigen of interest, such as a polypeptide (e.g., a protein). For example, the full-length polypeptide of a protein of interest can be a polypeptide containing the complete amino acid sequence of the protein of interest, as encoded by the mRNA encoding the naturally-occurring version of the protein. The antigen of interest can include, for example, a soluble protein (e.g., a cytoplasmic protein or secreted protein), a membrane-bound protein (e.g., a cell surface protein or an organelle membrane-bound protein), a misfolded protein (e.g., a prion), or any other polypeptide known in the art. In some instances, a full-length antigen can be fused to another polypeptide, such as a selectable marker or an epitope tag. A fragment of a full-length antigen can be used as a partial antigen in the methods described herein.

The term “target polypeptide,” as used herein, refers to any polypeptide of interest containing one or more epitopes to which a binding moiety, such as an antibody or antibody fragment, may bind. A target polypeptide can be, for example, a full-length antigen or a partial antigen.

By “binding moiety” or “affinity reagent” is meant any molecule capable of binding to another molecule (e.g., a target polypeptide). Binding moieties of the invention can include, for example, antibodies, antibody fragments, and antibody derivatives, such as those well known in the art. Exemplary antibodies and antibody fragments include IgGs, single-chain variable fragments (scFvs), and Fabs.

By the term “detectable gene” or “selectable marker” is meant any genetic indicator that can occur or be encoded by a nucleic acid and that allows for selection, screening, or detection. For example, a detectable gene may encode a protein that permits a cell to grow in a particular media (e.g., URA3), or a protein that imparts a detectable property such as fluorescence (e.g., GFP, YFP, CFP, dsRed, mCherry, or any other fluorescent protein known in the art) or development of a particular compound or color (e.g., LacZ), or a peptide that can be detected using a binding moiety (e.g., an antibody). In some instances, the detectable gene can include, for example, an epitope that can be bound by an antibody for detection using techniques such as, for example, immunofluorescence, fluorescence-activated cell sorting (FACS), flow cytometry, Western analysis, and/or immuno-histochemistry. A detectable gene can be selectively expressed in a cell only under certain conditions, such as, for example, if a particular promoter is activated (e.g., by a transcription factor and/or by the association of an activating domain and DNA binding domain).

As used herein, “bi-functional vector” means a nucleic acid vector containing a gene and elements permitting expression of the gene in at least two distinct cells. For example, a bi-functional vector may be used to express a gene of interest in a yeast (e.g., Saccharomyces cerevisiae) and a bacteria (e.g., E. coli). In some instances, the gene can be expressed in distinct forms depending on the cell in which the bi-functional vector is present. In certain instances, the protein product of the gene can be fused to a second protein in one cell type, and can alternately be fused to a third protein in the other cell type. For example, the gene of interest can be fused to the viral protein gp3 when expressed in E. coli, but can be fused to, e.g., a transcriptional activation domain or DNA binding domain when expressed in yeast. Thus, in some instances, the same bi-functional vector can be used for both bacterial expression in a phage display screen and for yeast expression in a yeast 2-hybrid assay. A vector (e.g., a bi-functional vector), or a portion thereof, can act as a “template” for expression of a gene, in that the nucleic acid sequence of the gene is present on the vector, such that an RNA polymerase can transcribe the nucleic acid sequence of the gene into an mRNA, which can subsequently be translated into protein. Alternatively, a gene can be expressed as a non-coding RNA, siRNA, shRNA, miRNA, piRNA, tRNA, or any other functional gene known in the art.

By “dsDNA” is meant a double-stranded DNA molecule, e.g., a DNA molecule containing two strands hybridized to each other. The two strands of a dsDNA molecule can be perfectly complementary, such that every base on each strand is bound to a base on the opposite strand. Alternatively, the two strands of a dsDNA molecule can include noncomplementary nucleotides. A “ssDNA” is a single-stranded DNA molecule, e.g., a single DNA strand that is not presently hybridized to another DNA strand. An ssDNA molecule can hybridize to another ssDNA molecule to form a dsDNA.

The term “antibody” is used herein in the broadest sense and specifically covers monoclonal antibodies, polyclonal antibodies, multispecific antibodies, and antibody fragments (e.g., scFvs and Fab fragments) so long as they exhibit the desired biological activity.

As used herein, “single-chain variable fragment” or “scFv” means an antibody fragment including the V_{H} and V_{L} domains of antibody, in which these domains are present in a single polypeptide chain. The Fv polypeptide can further include a polypeptide linker between the V_{H} and V_{L} domains, which can enable the scFv to form a desired structure for antigen binding. See, e.g., Pluckthun in The Pharmacology of Monoclonal Antibodies, Vol 113, Rosenburg and Moore eds. Springer-Verlag, New York, pp. 269- 315 (1994).

### BRIEF DESCRIPTION OF THE DRAWINGS

FIG. 1 is a flow diagram showing the high-throughput pipeline incorporating the Y2H assay during discovery screening. The Y2H addition to the pipeline, shown in green, can be placed, for example, between the second and third rounds of affinity selection by φD, as a means to identify affinity reagents against full-length proteins. The first φD discovery round can be used to enrich for low affinity binders in the library. The Y2H assay can then be used to enrich for binders against full-length, folded protein antigens. A second round of φD can be performed after AXM mutagenesis to affinity mature any potential affinity clones.

FIG. 2 is a diagram showing an exemplary cloning vector system, the pCH103 vector system. The pCH103 vector system has been constructed with the ability to function in both Y2H and E. coli-based φD methods. In E. coli, the protein expressed will depend on the E. coli genotype. (A) Due to the insertion of amber stop codon between the scFv and gp3, in non-suppressing E. coli, the expressed protein will be the scFv by itself. In suppressing strains of E. coli, the scFv will be fused to the gp3 protein. In yeast, the scFv can be fused to the yeast activation domain, or in alternate instances using a different vector system, to the DNA-binding domain. (B) Features of the pCH103 vector system include: (i) a GAL1 promoter used for the expression of genes cloned into pYESTrp2; expression is constitutive in L40 and inducible in EGY48/pSH 18-34, (ii) a V5 epitope, which allows detection of fusion protein(s) using the anti-V5 antibody, (iii) an SV40 large T antigen nuclear localization sequence (NLS), which localizes fusions to the nucleus for potential interaction with LexA fusions, (iv) a B42 activation domain (AD), a transcriptional activation domain that allows expression of reporter genes when brought into proximity with the LexA DNA binding domain (DBD) by two interacting proteins, (v) a CYC1 transcription termination signal, which permits efficient termination and stabilization of mRNA, (vi) a TRP1 gene, which permits auxotrophic selection of the plasmid in Trp⁻ yeast hosts (e.g., L40 or EGY48/pSH 18-34), (vii) 2-micron origin, for maintenance and high-copy replication in yeast, (viii) an f1 origin for rescue of single-strand DNA by a M13K07 helper phage, (ix) an encoded E. coli lac promoter (P_{E.coli}, for controlled expression of an M13 gp3 fusion construct), (x) an M13 gp3 gene, for φD fusions to the gp3 protein of M13, and (xi) a protein fusion site for cloning proteins into the multiple cloning site of the pCH103 vector.

FIG. 3 is a series of images showing features of the pCH103 vector system. (A) Test of amber suppression in E.coli: A version of the pCH103 vector was constructed that lacked a stop codon between the AD and Zeo genes. This construct was compared to a similar vector in which an amber stop codon was placed between the AD and Zeo genes. E. coli strains containing supE44 were shown to suppress the stop codon, allowing growth on plates containing zeocin. (B) Test of suppression in yeast: The same vector system as in FIG. 3A was tested in yeast. As expected, yeast was not able to grow on Zeo-containing plates if there was a stop codon 3′ of the AD gene. (C) Test of protein-protein interaction in Y2H through the translated recoded E. coli promoter peptides: An scFv against cMet was inserted into the vector and tested against cMet and 2 other unrelated bait-proteins in duplicate. The 3 promoters were tested for the ability of the recoded promoter peptide to not interfere with the interaction of the cMet scFv and the cmer bait. The tac and tacO promoters were shown to not interfere in the Y2H screen. The phoA promoter peptide, however, was found to interfere with the interaction of the bait and prey fusions. (D) Test of F1ₒᵣᵢ: M13K07 helper phage was infected into pJaneway-containing E. coli to create transducing lysates of packaged pJaneway plasmid DNA. These lysates were sterile-filtered and then used to transduce lawns of E. coli strain CJ236 on +Zeo plates to Zeo′.

FIG. 4 is a series of images showing that scFvs raised against protein fragments can recognize full-length (FL) proteins. (A) Purified ZNF384 fragments (amino acids 1-100) tagged with Maltose Binding Protein (MBP) tags and lysates of HEK293 cells transiently transfected with an expression construct for full-length ZNF384 (DNASU) were separated on an acrylamide gel, transferred to a nitrocellulose membrane and probed with FLAG-tagged scFvs raised against the ZNF384 fragment, followed by anti-FLAG-HRP secondary Ab to detect the scFvs. (B) HEK293 cells transiently transfected with full-length ZNF622 and ZNF384 were lysed to prepare lysate (L) and chromatin (C) fractions. The lysates and chromatin fractions were incubated with scFvs (raised against either amino acids 1-100 of ZNF384 or amino acids 37-139 of ZNF622) immobilized on FLAG beads. The beads were washed and the complexes were eluted with buffer containing 2% SDS. The eluted complexes were then analyzed by Western Blot with anti-V5-HRP antibody to detect the presence of full-length transcription factors in eluates (tagged with V5 tag). (C) This page shows the experiment performed as in FIG. 4B, but using formalin cross-linked cells following the ChIP protocol.

FIG. 5 is a diagram showing an scFv-to-IgG reformatting scheme based on AXM cloning. In this scheme, the variable heavy and light chain genes from enriched scFvs will be amplified using reverse primers containing phosphorothioate linkages on the 5′ end. The resulting double-stranded DNA is treated with T7 exonuclease to selectively degrade the unmodified strand of the dsDNA molecule. The resulting single-stranded DNA, or “megaprimer,” will then be annealed to a circular, single-stranded DNA containing the CMV promoter, IL2 signal sequences, internal ribosome entry site (IRES), and the light and heavy chain variable and constant domains with Eco29kl restriction sites in the CDR regions. Annealed megaprimers can be used to prime in vitro DNA synthesis by DNA polymerase. The resultant ligated, heteroduplex product is then transformed into E. coli AXE688 cells, which express the Eco29kl restriction endonuclease. Thus, the presence of Eco29kl in these cells favors survival of the newly synthesized, recombinant strand that incorporates the megaprimer.

FIG. 6 is a series of images showing the results of mating yeast strains transformed with either a vector encoding an scFv (or control) fused to a binding domain, or at least a portion of a target protein (Myb, EZH2, or a control) fused to an activation domain. (A) In the positive control reaction (Reaction 1 of Table 1); pGBKT7-p53 mated to pGADT7-T), growth was observed on all minimal media, indicating a strong interaction. (B) In the negative control reaction (Reaction 2 of Table 1; pGBKT7-Lam mated with pGADT7-T), no growth was observed, as expected. (C) In reaction 8 of Table 1 (pGBKT7-AXM1387 mated with pGADT7-Myb20), growth was only observed on TDO, indicating a weak interaction. (D) In reaction 15 of Table 1 (pGBKT7-AXM1389 mated with pGADT7-Myb100), growth was seen on all minimal media, indicating a strong interaction. (E) In reaction 17 of Table 1 (pGBKT7-AXM2274 mated with pGADT7-EZH2100), growth was seen only on TDO, indicating a weak interaction.

FIG. 7 is a series of images showing the results of mating yeast strains transformed with either a vector encoding a binding moiety (an scFv, known binding partner, or other control) fused to an activation domain, or at least a portion of a target protein (GRAP2, XIAP, LNMA, or a control) fused to an binding domain. The results show that GRAP2 interacts with its known binding partner, LCP2 (positive control), but not to CASP9 (negative control), in the yeast two-hybrid system. In addition, GRAP2 was shown to interact with scFv1, which was selected against the GRAP2_2 peptide shown in Table 3. Further, one of the scFvs tested, AXM1389, showed interaction with full-length GRAP2.

### DETAILED DESCRIPTION OF THE INVENTION

The present invention provides methods for the high-throughput (HT) validation of recombinant antibodies (rAbs) against full-length (FL) human cytoplasmic and nuclear proteins, in which the rAb was generated against either partial fragments of the antigen (partial-Ag), or even synthetic peptides. For example, such methods can involve performing a yeast two-hybrid (Y2H) assay using the full-length antigens (FL-Ag) to validate rAbs generated by, for example, phage display (φD). The methods of the invention can be incorporated into any high-throughput antibody-generating pipeline, and can eliminate the need for lengthy, laborious and low-throughput characterization of individual affinity reagents. The invention further features a vector system that enables both Y2H and φD without the requirement of any subcloning (e.g., the pCH103 vector system). The invention also features an rAb framework (e.g., the B2A framework) that can function both in φD (e.g., when fused to the phage M13 gp3 protein) and in Y2H (e.g., when fused to a yeast DNA binding domain or activation domain, such as the B42 activation domain). These methods permit the rapid, efficient, and low-cost production and validation of highly specific antibodies with strong binding affinity for a target polypeptide of interest.

### Antibody Screening Methods

The present invention provides methods of screening affinity reagents (e.g., antibodies) capable of binding to a desired target polypeptide from a library of such affinity reagents. In some instances, a screen is performed using phage display (φD), in which each of the antibodies is expressed on the surface of a virus (e.g., M13 phage) and then exposed to an antigen representing to the target polypeptide (e.g., a full-length antigen or partial antigen). Bound virus can then be isolated from unbound virus, thereby selecting for viruses expressing antibodies capable of binding to the antigen. φD methods are well known in the art, and can include all-liquid φD methods.

In some instances, antibodies can be selected by the methods of the present invention using partial antigens. Such antibodies can desirably bind to full-length target polypeptides at, for example, minimum desired affinities. Use of partial antigens can complement the use of properly folded full-length polypeptides as targets. For example, affinity reagents may be desired against post-translationally modified molecules (e.g., phosphorylated targets). Many such target sites are located within unstructured regions of the target polypeptide, such that the partial antigen is a good model of the epitope. A particular full-length protein may, in some instances, be difficult to purify. It may also be desirable to distinguish between members of a target protein family. In such cases, selection is preferably focused on the sequence region where the family members differ, and subtractive approaches may not always be successful. Thus, targets may consist of synthetic peptides (e.g., synthetic peptides of less than 40 amino acids) or protein fragments (e.g., protein epitope signature tags, or PrESTs, having lengths, for example, of greater than 40 amino acids), for which folding may be partial, but which may still contain several epitopes of the full-length protein. For such protein fragments, the genetic sequences can be, for example, fused to a solubility/folding partner. This approach can yield a high success rate (e.g., >90%) in producing soluble fusion-product, which can be subsequently purified. The resultant purified fusion product can be used to generate recombinant antibodies (rAbs) against the partial antigen portion of the fusion, and these rAbs can have a high success rate at recognizing the full-length antigen.

The present invention also features the screening and validation of rAbs against full-length post-translationally modified proteins. In some instances, the full-length protein can be post-translationally modified, for example, by co-expressing a kinase or other modifying enzyme as known in the art in yeast during Y2H validation. In certain instances, site-specific modification of the antigen can be performed, for example, by use of a suppressing host. For example, the pSer system can be used in E. coli (e.g., in a bacterial two-hybrid system), or iodo-tyrosine can be incorporated in a two-hybrid system (e.g., yeast two-hybrid or bacterial two-hybrid). Any other methods for suppression in yeast or bacteria may also be useful for this purpose.

In one embodiment, the tyrosyl-tRNA Synthetase (TyrRS) of E. coli is modified to recognize and incorporate phosphotyrosine at amber stop codons placed at specific positions within a gene sequence. In an alternate example, phosphoserine (pSer) can be incorporated into full-length protein [Dieter Soll, Yale University, see also M. Weiner, additional support]. The pSer system can be used to generate site-specific phosphoserine full-length proteins that may be tested, for example, in a bacterial two-hybrid (B2H) system (Battesti et al., Methods 58(4):325-34, 2012; Dove et al., Methods Mol Biol. 261:231-46, 2004; Velasco-Garcia et al., 58(11):1241-57, 2012) in a pSer-suppressing E. coli host cell.

In some instances, affinity reagents determined to be capable of binding to a target polypeptide according to the methods described above can be identified by isolating the nucleic acid encoding the affinity reagent (e.g., a vector such as those described herein) and sequencing the isolated nucleic acid. Methods of sequencing are well known in the art, including methods of deep sequencing and NextGeneration sequencing. Sequencing can include, for example, Sanger sequencing or next generation sequencing technologies. Exemplary next generation sequencing technologies include, without limitation, Hyseq2500, Ion Torrent sequencing, Illumina sequencing, 454 sequencing, SOLiD sequencing, and nanopore sequencing. Additional methods of sequencing are known in the art.

### Validation Screens

### Yeast 2-Hybrid Validation

The present invention features yeast 2-hybrid (Y2H) as a validating counter-screen for antibodies identified as capable of binding to a target polypeptide of interest (e.g., using a phage display as described herein). A pool of such identified antibodies can thus be enriched for the antibodies having the highest binding affinity to the target polypeptide by the Y2H counter-screen. The Y2H system is ideal for this purpose for several reasons, including (i) its ease-of-use; (ii) its capacity for automation (e.g., in 96-well microtiter plates; see, e.g., [Buckholz, Slentz-Kesler]); and (iii) its suitability for the gene constructs such as those described herein.

Y2H is well known in the art as a technique useful for identifying and/or characterizing protein-protein interactions that occur within a cell. Briefly, Y2H involves the fusion of one polypeptide of interest to the activation domain of a transcription factor (the “prey”), and the fusion of a second polypeptide of interest to the DNA binding domain of the transcription factor (the “bait”). In some instances, the activation domain is a B42 activation domain, and/or the DNA binding domain is a GAL4 DNA binding domain. The two fusion proteins can then then expressed in a yeast cell together. For example, one fusion protein can be expressed in yeast mating type a and the other fusion protein can be used in yeast mating type a. The two haploid yeast strains can then be mated to produce a diploid yeast expressing both fusion proteins. Generally, the bait will bind to a DNA promoter or enhancer element controlling the expression of a detectable gene, but will not be capable of activating expression of the detectable gene alone. If the two fusion proteins do not bind, then the DNA binding domain and activation domain will remain separate and expression of the detectable gene will not occur. If, however, the two fusion proteins bind to each other, then the activation domain and the DNA binding domain are brought into close enough proximity to each other such that the activation domain will be able to initiate expression of the detectable gene. As such, binding between the two polypeptides of interest can be determined based on the expression of the detectable gene.

Detectable genes useful in the validation methods of the present invention are well known in the art. Exemplary detectable genes include, for example, genes that enable cell growth in a particular media (e.g., URA3), genes encoding fluorescent proteins (e.g., GFP, YFP, CFP, dsRed, mCherry, or any other fluorescent protein known in the art) or enzymes that can produce a colorimetric readout (e.g., LacZ), or any other polypeptide marker or label as known in the art. In some instances, a detectable gene can be any gene for which expression level can be detected. For example, mRNA expression of a gene can be determined using techniques such as quantitative real-time PCR, Northern analysis, or RNA Seq. The expression of such detectable genes can be detected according to methods well understood in the art.

In addition to cytoplasmic proteins, Y2H has also been used to characterize the cytoplasmic domains of membrane-bound proteins (Brückner et al., Int J Mol Sci. 10(6):2763-88, 2009). To rapidly elucidate the function of many genes as they are identified by large scale sequencing efforts, systems have been developed for HT cloning, genotyping, and yeast two-hybrid analysis [(HT-Y2H), (Buckholz et al., J Mol Microbiol Biotechnol. 1(1):135-40, 1999; Chen et al., Genome Res. 10(4):549-57, 2000; Taylor et al. Biotechniques 30(3):661-6, 2001; Uetz et al., Nature 403(6770):623-7, 2000; Uetz et al., Curr Opin Microbiol. 3(3):303-8, 2000; Walhout et al., Yeast 17(2):88-94, 2000). These systems make possible massively parallel analysis of both genome and proteome. Y2H can be used, for example, to identify links between uncharacterized proteins and known pathways, which can be useful, for example, for dissecting the molecular networks operating in cells.

It is contemplated that low affinity rAbs from early phage display (φD) selection rounds can be validated in yeast according to the methods of the invention, which is desirable for a number of reasons. First, as is well known in the art, Y2H is extremely sensitive and can detect interactions between partners having a K_{D} substantially greater 300 nM [Rid]. Second, as is well known in the art, even small amounts of expressed proteins having very weak affinities for each other can be sufficient to elicit a positive Y2H response (Golemis et al., Curr Issues Mol Biol. 1(1-2):31-45, 1999; Estojak et al., Mol Cell Biol. 15(10):5820-9, 1995; Rid et al., Assay Drug Dev Technol. 11(4):269-75, 2013; Rajagopala et al., Methods Mol Biol. 781:1-29, 2011). Third, the rAb HT-pipeline described herein is robust and able to isolate affinity reagents against peptides and protein fragments. It has been shown, using three-dimensional co-crystal structures available for protein complexes in the Protein Data Bank (Berman et al., Nucleic Acids Res., 28: 235-242, 2000) and for domain-domain interactions (Stein et al., Nucleic Acids Res. 39 (Database issue): D718-D723, 2011), that Y2H binary interactions reflect direct biophysical contacts. Y2H sensitivity can also correlate with the number of residue-residue contacts, and thus with interaction affinity.

The Y2H system can also be used for discovery of affinity reagents. However, the comparatively inefficient transformation efficiency of yeast can limit the size of yeast libraries (generally <10⁸ antibodies). As such, yeast are more suitable for the validation round after, for example, a φD or ribosomal display enrichment screen. Bacterial 2-hybrid and mammalian two-hybrid systems are also envisioned as alternative methods for antibody discovery and/or for validation of already-identified antibodies.

### Immunoprecipitation Validation

As an additional quality control check after rAb generation, affinity-matured lead candidate rAb molecules can be further validated using immunoprecipitation (IP), for example, using FLAGTM-tagged full-length proteins expressed in, e.g., transiently-transfected mammalian cells. Immunoprecipitation methods useful for this validation are well known in the art.

### Antibody Libraries

The present invention features methods for identifying, from a library of antibodies, particular antibodies capable of binding to a target polypeptide of interest, and validating the antibodies identified thusly. Such antibody libraries can be constructed according to methods well understood in the art. An exemplary constant framework antibody library useful in the methods of the invention can, for example, involve encoding triplet-codon mutagenesis at 18 different positions within the six complementarity determining regions (CDRs). In some instances, this can involve an NNK codon within the 18 positions with the six CDRs of the antibody. Exemplary antibodies for use in the methods of the invention include scFvs, IgGs, Fab fragments, and any other antibody or antibody fragment as known in the art. In some instances, the antibodies can include scFvs based on the B2A framework. In certain instance, the B2A frarmework includes a nucleic acid sequence having at least 80% sequence identity (e.g., at least 80%, 85%, 90%, 95%, 96%, 97%, 98%, or 99% sequence identity) to the following sequence:

(SEQ ID NO: 1)
DYKDDDDKLSYELTQPPSVSVSPGQTARITCSGDALPKKYAYWYQQKSGQ
APVLVIYEDSKRPSGIPERFSGSSSGTMATLTISGAQVEDEADYYCYSTD
SSGNYRVFGGGTKLTVLGEGKSSGSGSESKASEVQLVQSGADVKKPGASL
TISCQASGYTFTDYWISWVRQKPGKGLEWMGRIDPSDSYTNYGPSFQGHV
TISADRSTATAYLQWRSLEASDTAMYYCVRSSMVTSGGTIPEYYQHWGPG
TLVTVSS

In one instance, the B2A framework includes a nucleic acid sequence identical to SEQ ID NO: 1.

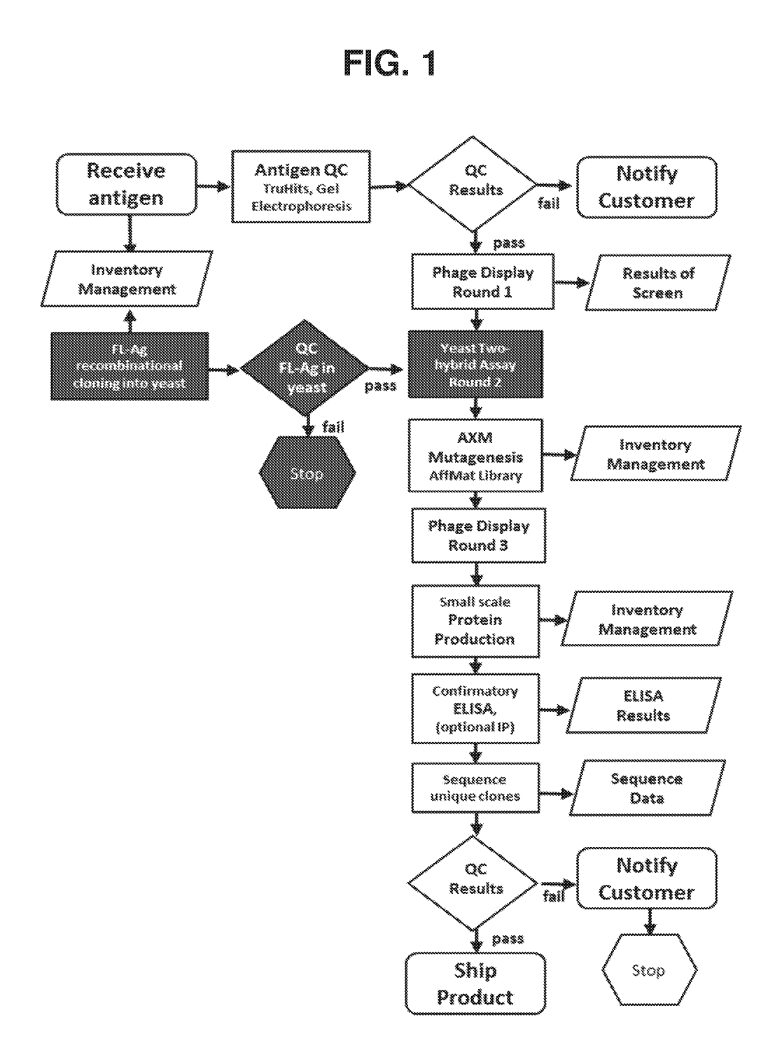

In some instances, an antibody library includes variants of a particular antibody. Such libraries of antibody variants can be generated, for example, using molecular evolution methods such as those described in PCT Application No. PCT/US2014/068595 and PCT Publication No. WO 2014/134166 (incorporated herein by reference in their entirety). An antibody identified as capable of binding to a target polypeptide of interest (e.g., according to the methods of the invention) can be further optimized for strength of binding to the target polypeptide by, for example, performing an additional round of molecular evolution followed by screening and validating the resultant variants for improved binding affinity to the target polypeptide. This process can be repeated, for example, until an antibody variant having a desired binding affinity to the target polypeptide is identified and validated (see, e.g., FIG. 1).

Each individual antibody of an antibody library can be contained, for example, in a vector, such as a bi-functional vector that enables expression of the antibody in multiple cell types. For example, the antibody can be present in a bi-functional vector that permits expression of the antibody in both bacteria (e.g., E. coli) and a eukaryotic cell (e.g., yeast or a mammalian cell). Moreover, the bi-functional vector can allow for fusion of the antibody to particular polypeptide domains depending on the cell type (or even the particular strain) in which it is expressed. For example, the pCH103 vector described herein permits expression of a fusion protein including the antibody and gp3 in E. coli, thus enabling the use of the antibody in phage display. In yeast, the pCH103 vector permits expression of a fusion protein including the antibody and the B42 activation domain, thus enabling the use of the antibody in yeast 2-hybrid. In addition, the pCH103 vector includes an amber stop codon between the antibody and the gp3 gene, such that the fusion protein is only expressed in E. coli strains capable of suppressing the amber stop (e.g., E. coli strains expressing supE44). Vectors useful in the invention can further include, for example, additional genes (e.g., selectable markers), promoter and enhancer elements, and further polypeptide domains fused to the antibody of interest, such as epitope tags).

### Antigens

The present invention features the production, screening, and validation of antibodies directed against particular target polypeptides or antigens. The binding between an antibody and a target antigen occurs at one or more epitopes, which can be defined by, for example, the position and polarity of amino acid residues in a particular region of the target antigen. Such epitopes can, in some instances, be maintained in peptide fragments (e.g., partial antigens) of the target antigen at sufficient similarity to the analogous epitope on the full-length antigen, such that an antibody capable of binding to the epitope present in the peptide fragment can also bind to the analogous epitope on the full-length antigen. Thus, it is contemplated that antibodies of the invention can be screened against partial antigens or full-length antigens. Likewise, the antibodies can be validated against partial antigens or full-length antigens. For example, antibodies can be screened against partial antigens and validated against full-length antigens.

Antigens useful in the methods of the invention can be produced according to methods well known in the art. For example, antigens can be expressed in cells by transforming a cell with a nucleic acid encoding the antigen, and inducing the cell to transcribe and translate the nucleic acid into the amino acid sequence of the antigen. Alternatively, antigens (particularly partial antigens, e.g., partial antigens containing 40 amino acids or fewer) can be synthesized using peptide synthesis methods well understood in the art. Antigens can be associated with binding moieties, to allow a plurality of antigens to be readily isolated from a mixture. For example, antigens can be bound or fused to biotin, such that the antigens can be isolated using streptavidin or NeutAvidin, e.g., attached to a surface (e.g., a well, tube, strip, or bead).

The following examples are intended to illustrate, rather than limit, the invention.

### EXAMPLES

### Example 1

### Materials and Methods

### Bacterial Strains and Vectors

The E. coli strain, TG1 [F′ (traD36, proAB+ lacl^{q}, lacZΔM15), supE, thi-1, Δ(lac-proAB), Δ(mcrB-hsdSM)5, (rK⁻mK⁻)], purchased from Lucigen (Middleton, Wis.), was used to develop the E. coli strain AXE688, by transforming TG1 with the pAX1492 plasmid that encodes the Eco29kl RM operon. The E. coli strain CJ236 (FΔ(HinDIII)::cat(Tra⁺, Pil⁺, Cam^{R})/ung-1, relA1, dut-1, thi-1, spoT1, mcrA) was purchased from New England BioLabs (NEB; Waverly, Mass.). Electrocompetent and chemically competent strains will be made following NEB protocols. The template plasmids for AXM mutagenesis will be derivatives of the phagemid pCH103, with the same scFv template genetically fused to the coat protein gp3 of bacteriophage M13. Each of the 6 CDRs of this template scFv will be modified to contain both opal (TGA) stop codons and Eco29kl restriction endonuclease recognition sites. In this template, non-recombinant clones are non-functional with respect to display of the scFv. The opal stop codons and Eco29kl restriction endonuclease sites will be absent in fully recombinant rAbs.

### All-Liquid φD

A phage library including approximately 1×10¹⁰ synthetically diversified Abs, all based on single V_{H} and Vₖ domains will be used. Phage displaying the scFvs will be prepared by growth of aliquots of bacteria from each library followed by superinfection with the helper phage, KM13 (Kristensen et al., Nucleic Acids Res. 15(14):5507-16, 1987). A multi-well plate will be coated with NeutrAvidin (Thermo Fisher Scientific, Rockford, Ill.). After washing with PBS, wells will be blocked with 2% nonfat dry milk in PBS (MPBS). To capture a biotinylated peptide on the NeutrAvidin-coated wells, the wells will be washed and incubated with the peptide at room temperature. Phage (around 1×10¹³ transducing units) from the library will be added to the wells and incubated at room temperature. Bound phage will be eluted by addition of trypsin (Sigma-Aldrich, St. Louis, Mo.). The eluate will be used for infection of exponentially growing E. coli TG1 in liquid and grown overnight. An aliquot will be used for phage rescue; glycerol will be added to the remainder and the suspension stored at −80° C. Phage will be titered. Second and third rounds will be carried out as above for selection of clones of high affinity and specificity. We will use multi-well plates and screen partial Ags in several wells. The wells will be kept independent. An enzyme linked immunosorbent assay (ELISA) will be used to identify binding phage clones, and DNA sequencing will be used to identify unique clones.

All-Liquid Y2H Using a- and α-Mating Type Strains of Yeast and Testing scFvs in φD

We will use the all-liquid Y2H assay invented by the Weiner group (Buckholz et al., supra). We will incorporate the gene, URA3 (orotidine-5′ -phosphate decarboxylase), as a counter-selectable marker. The endogenous URA3 gene will be inactivated, and a reporter cassette introduced in which expression of URA3 is dependent on the presence of a bait/prey interaction. If URA3 is expressed, the uracil biosynthesis pathway will be functional, and 5-fluoroorotic acid (5-FOA), which is added to the media, will be metabolized into a suicide substrate for the essential thymidylate synthase enzyme.

### Immunoprecipitation (IP)

Immunoprecipitation will be performed by first establishing the FLAG-tagged protein production in the cell line of choice (e.g., CHO, 293T, or HeLa) after transient transfection. For the isolation of cytoplasmic and nuclear proteins, depending on expression levels, 10⁶-10⁷ cells will be resuspended in RIPA buffer (25 mM Tris-HCl (pH 7.6), 150 mM NaCl, 1% NP-40, 1% sodium deoxycholate, 0.1% SDS) for 20 minutes and the clarified supernatant resulting from centrifugation will be moved to a clean tube. For nuclear-localized proteins, 10⁷ cells will be gently resuspended in ice-cold hypotonic buffer and incubated on ice. The cells will be homogenized, and the nuclear component, after centrifugation, will be resuspended in RIPA buffer. For IP, bovine serum albumen (BSA)-blocked anti-FLAG beads will be mixed with the cell lysate prepared as described and incubated with gentle agitation. The desired scFv will be added to the slurry, incubated, and then washed. After the last wash, the beads will be boiled, centrifuged, and the clarified lysate loaded onto a polyacrylamide gel for subsequent Western analysis. The proteins we currently transiently transfect have V5 tag. The lysates are usually mixed with a scFv ON and the FLAG beads are added next day for 2 hr. The FLAG beads capture a scFv (which is FLAG-tagged) and the protein bound to it (if any). The beads are then extensively washed and eluates are analyzed by WB with anti-VS-HRP antibody to detect the presence of full-length antigen.

Construction of scFv Library in pCH103

We have previously constructed several recombinant scFv libraries having >10¹⁰ diversity using our novel method for the in vivo restriction of non-recombinant DNAs (see, e.g., Holland et al., J. Immunol. Methods, in press). We have successfully used these libraries to identify scFvs against >100 different FL-proteins, protein fragments, and short peptides. Our typical scFv library uses a constant-framework with amino acid changes limited to the CDRs. The scFvs are displayed on the surface of bacteriophage M13 as genetic fusions to coat protein gp3. We will use our novel library-construction methodology for the construction of the pCH103 library.

### Affinity Determination by AlphaScreen

A constant concentration of purified scFv is incubated with a constant concentration of biotinylated Ag and the streptavidin donor and FLAG acceptor AlphaScreen beads (Perkin Elmer). An increasing concentration of non-biotinylated antigen is added, and the competitor concentration at which half the maximum signal is generated (IC₅₀) is estimated to be the K_{D}.

### Protein Expression and Purification

The scFvs will be expressed in E. coli Mach1 cells (Life Technologies) in low phosphate media supplemented with ampicillin (100 μg/ml). Baffled flasks at a maximum 20% of flask volume will be used to ensure good aeration. The culture pellets will be stored frozen at −80 ° C. Cell pellets will be resuspended in BugBuster (EMD Chemicals, Gibbstown, N.J.) supplemented with Benzonase (EMD Chemicals, Gibbstown, N.J.), phenylmethanesulfonylfluoride (PMSF), and protease inhibitor cocktail. The resuspended pellets will be clarified by centrifugation and the scFvs will be purified from the cleared lysates on HisPur™ Cobalt resin (Thermo Scientific, Rockford, Ill.). After binding the soluble scFv protein, the columns will be washed and the protein will be eluted by the addition of imidazole and analyzed by SDS-PARTIAL-AGE. HiPrep 26/10 Desalting columns (GE Healthcare, Piscataway, N.J.) will be used for buffer exchange to PBS.

### IgG Antibody Production

Freestyle CHO or 293F cells (Life Technologies) will be transiently transfected with the IgG constructs using Freestyle MAX reagent (Life Technologies) in 30-ml batches. This culture size has been shown to be sufficient for achieving yields of 100 μg of soluble IgG antibodies, which is sufficient material for initial characterization. The rAb IgGs will be purified in high-throughput using a protein A affinity resin. We typically recover >80% of the soluble IgG with >90% purity in a single-step purification. Endotoxin levels in our purified IgGs are expected to be less than 50 endotoxin units (EU) per milligram based on a standard Limulus amebocyte lysate (LAL) test (Sharma, Biotechnol Appl Biochem. 8(1):5-22, 1986; Tuomela et al., Gene Ther. 12 Suppl 1:S131-8, 2005; Vanhaecke et al., J Clin Pharm Ther. 12(4):223-35, 1987). We will conduct a time-course to determine the best post-transfection time to harvest the protein.

### Example 2

### Overview of Antibody Framework and Library Design

We have identified a constant framework scFv-based Ab library encoding an NNK codon (see, e.g., Benhar, Expert Opin Biol Ther. 7(5):763-79, 2007; Chan et al., Int Immunol. 26(12):649-657, 2014; Miersch et al., Methods. 57(4):486-98, 2012; Mondon et al., Front Biosci. 13:1117-29, 2008; Tohidkia et al, J Drug Target. 20(3):195-208, 2012) at 18 different positions within the six complementarity determining regions (CDRs) (Mandrup et al., PLoS One. 8(10):e76834, 2013). The B2A framework for the library identified and used in these experiments was selected based on its capacity for functionality in both phage display (φD), when fused to the M13 gp3 protein, and in yeast 2-hybrid (Y2H), when fused to the activation domain. Notably, although the rAb-generating platform of the present invention is currently used with single-chain variable fragments (scFvs), the platform is readily applicable to other applications, such as, for example, Fab, IgG, and yeast-display libraries. For example, high-throughput conversion of scFvs to full IgGs can be integrated within the pipeline.

Identification of an Ab Framework that Works in Both Phage Display and in Yeast Cytoplasm

To identify a suitable framework for our Y2H and φD library, we screened several hundred scFv frameworks from a naïve cDNA library, and identified one framework, which we labeled B2A, that worked exceptionally well in both φD and within the cytoplasm of yeast. The B2A framework could easily be both expressed in and purified from E. coli, and was thus chosen as the framework from which our initial NNK-based constant-framework φD library would be constructed. These useful properties of the B2A framework are notable, as a majority of scFvs do not work as intrabodies due to improper folding of the disulfide bonds in the reducing environment of the cytoplasm.

To further validate the B2A framework, we tested large-scale purification and were able to demonstrate an ability to purify 5-10 mg of scFv per liter of E. coli culture with >90% purity. This means that 0.3 liters of E. coli culture should be sufficient to generate enough purified material for rAb evaluation. For down-stream processing, we converted B2A into IgG molecules and showed that the resultant IgGs are strongly expressed in CHO cells and are functional when subsequently isolated from the cells.

### Library Design, Construction and Diversity Using the AXE688 Strain

For antibody library construction, we have utilized a method using in vivo Eco29kl restriction endonuclease digestion to eliminate non-recombinants, as we have described in, for example, PCT Application No. PCT/US2014/068595 and PCT Publication No. WO 2014/134166 (each of which is incorporated by reference herein in its entirety). The library diversity for libraries generated using this method was estimated at >10¹⁰. This is, in actuality, 2-10× better than the diversity for libraries using standard phage display methods that are referred to as “>10¹⁰” libraries, as traditional phage display methods yield libraries that are <40% recombinant. By contrast, libraries constructed used our method were >99% recombinant. This high recombinant percentage occurred because: (i) electrocompetent cells can take up more than one plasmid molecule per transformation event, (ii) we used 10× saturating amounts of plasmid DNA to transform our cells, and (iii) our AXE688 strain self-selects against non-recombinants because non-recombinants retain at least one Eco29kl site in one or more CDRs. When quality control analysis was performed on our libraries, we typically found that >99% of the scFv samples sequenced encode full-length scFvs.

### Example 3

### The pCH103 Cloning Vector System can Eliminate the Need for Subcloning Proteins for Testing in φD, Yeast Two-Hybrid, and E. coli Protein Expression

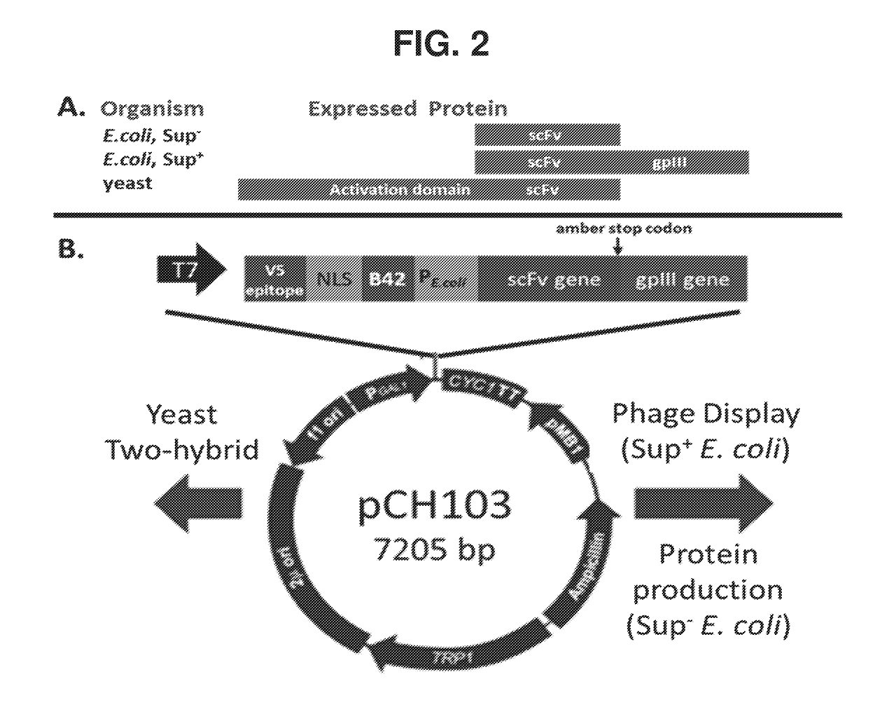

Technologies for Y2H and φD can be leveraged to develop a selectable and scalable system to obtain, identify, and affinity mature antibodies in an extremely high throughput and cost effective manner. A bi-functional vector system (pCH103) has been constructed with the capability to function in both Y2H and E. coli-based φD methods (FIG. 2). In E. coli, the protein expressed will depend on the E. coli genotype. Major features of the pCH103 vector system, and benefits thereof, are described below.

pCH103 Vector Design

As shown in FIG. 2A, an amber stop codon is inserted between the protein of interest (in this case, an scFv) and gp3. As a result, in non-suppressing E. coli, the expressed protein will be the scFv by itself. In suppressing strains of E. coli, the scFv will instead be fused to the gp3 protein. In yeast, the scFv will instead be fused to the yeast activation domain (or alternatively, to the DNA-binding domain). As shown in FIG. 2B, the pCH103 vector system includes the following features:
(i) GAL1 promoter, used for the expression of genes cloned into pYESTrp2. Expression is constitutive in L40 and inducible in EGY48/pSH 18-34.
(ii) V5 epitope, which allows detection of fusion protein(s) using the Anti-V5 Ab
(iii) SV40 large T Ag nuclear localization sequence (NLS), which localizes fusions to the nucleus for potential interaction with LexA fusions
(iv) B42 activation domain (AD), a transcriptional activation domain that allows expression of reporter genes when brought into proximity with the LexA DNA binding domain (DBD) by two interacting proteins
(v) CYC1 transcription termination signal, which permits efficient termination and stabilization of mRNA
(vi) TRP1 gene, for auxotrophic selection of the plasmid in Trp⁻ yeast hosts (e.g., L40 or EGY48/pSH 18-34)
(vii) 2 micron origin, for maintenance and high-copy replication in yeast
(viii) f1 origin, for rescue of single-strand DNA by M13K07 helper phage
(ix) Encoded E. coli lac promoter (P_{E.coli}, controlled expression of M13 gp3 fusion construct)
(x) M13 gp3 gene, for φD fusions to the gp3 protein of M13
(xi) Protein fusion site (site where proteins are cloned into the multiple cloning site of the pCH103 vector)
   Test of Amber Suppression in E. coli

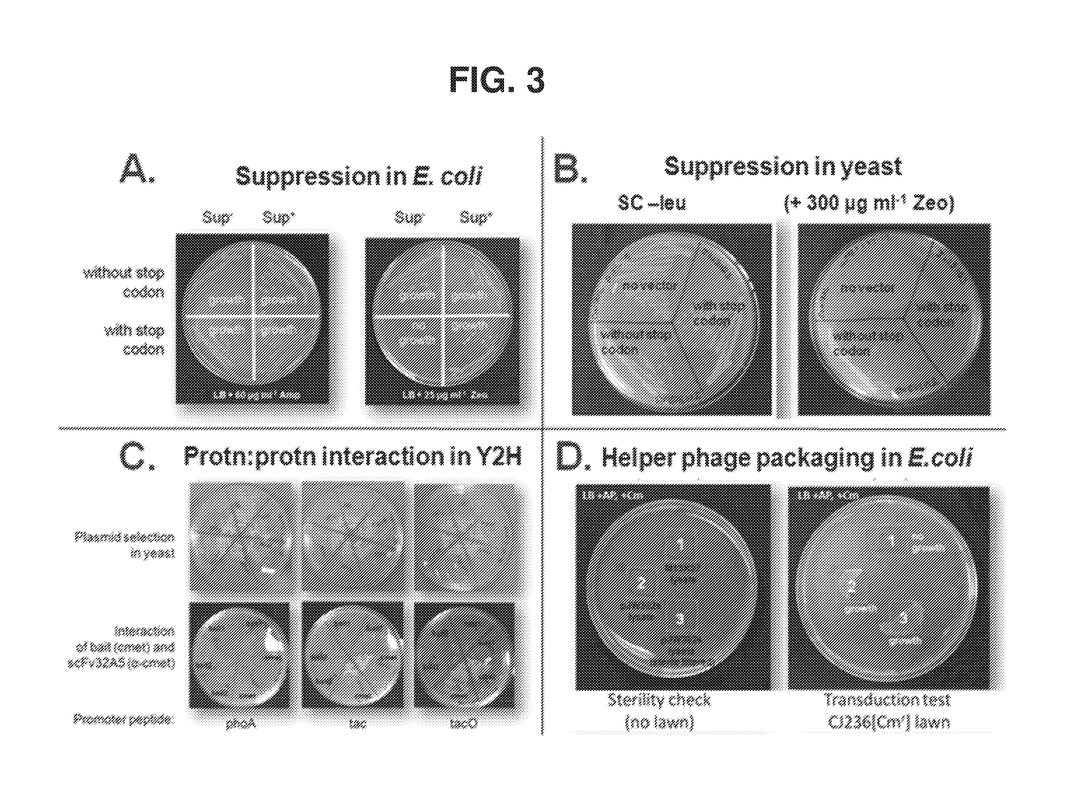

In order to test the amber stop codon placed between the scFv and the gp3 genes (FIG. 3A), a zeocin resistance (zeoR) gene was cloned into a derivative of pCH103 in place of the gp3 gene. A construct of this modified vector was made that did not have a stop codon between the B42 AD and ZeoR genes. This construct was compared to a similar vector in which an amber stop codon was placed between the two genes. E. coli strains containing supE44 were shown to suppress the stop codon, allowing growth on plates containing zeocin. Strains of E. coli unable to suppress an amber stop codon failed to grow on plates containing the zeocin antibiotic.

### Test of Amber Suppression in Yeast of an Amber Stop Codon Placed Between the B42 AD and the ZeoR-Genes

The same vector system as above, in which zeoR was cloned in place of gp3, was tested in yeast (FIG. 3B). Yeast cells cannot suppress amber stop codons. Thus, as expected, yeast cells were not able to grow on Zeo-containing plates if there was an amber stop codon placed 3′ of the AD gene. By contrast, if the amber stop codon was deleted, yeast were able to grow on zeocin-containing plates.

Demonstration of: a Functional Promoter Linker-Peptide Sequence, Corresponding to an E. coli Promoter Element, Placed Between the Y2H B42 AD and an scFv; and the 82A Framework Working in Yeast

An scFv against the human protein cMet was inserted into the pCH103 vector and tested against a cMet-DBD fusion along with 2 other unrelated bait-proteins. Three E. coli promoters (phoA, tac and tacO) were fused to the 3′ end of the yeast AD gene such that the E. coli promoters doubled as an open reading frame linker peptide that was inserted between the carboxyl-terminus of the yeast AD and the amino-terminus of the anti-cMet scFv. The phoA, tac and tacO promoters were each separately tested for the ability of the respective promoter linker-peptide to not interfere with the interaction of the anti-cMet scFv with the FL-cMet bait (FIG. 3C). The tac and tacO promoters were shown not to interfere in the Y2H screen. The phoA promoter peptide, however, was found to interfere with the interaction between the bait and prey fusions.

### Test of F1ₒᵣᵢ

M13K07 helper phage was used to infect E. coli cell, containing the pCH103-derivative plasmid (pJW302s), and produce transducing lysates of packaged ssDNA pCH103 plasmid DNA. These lysates were sterile-filtered and then successfully used to transduce an E. coli strain on agar plates containing zeocin to zeoR (FIG. 3D).

### Example 4

### Peptides and Protein Fragments can be Successfully Utilized to Generate IP-Grade scFvs

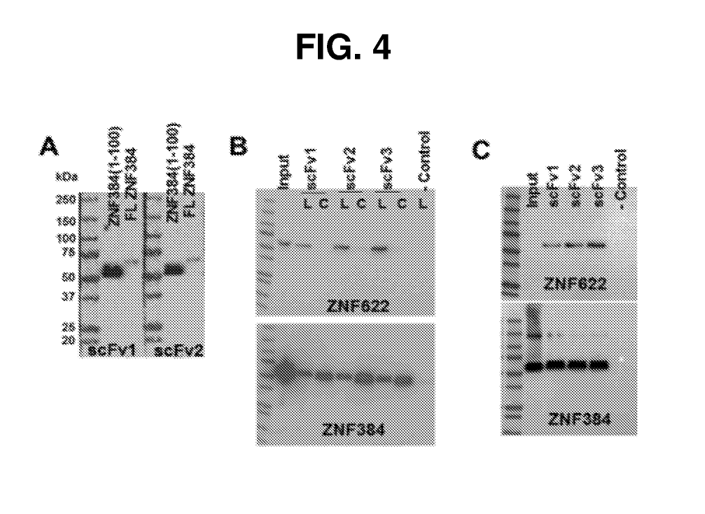

Expression and purification of sufficient quantities of full-length proteins often presents an obstacle in recombinant antibody generation. In the case of transcription factors (TFs), high homology between domains (i.e. DNA binding domains) and members of the same protein family can also compromise antibody specificity. To systematically isolate anti-TF antibodies by φD, we utilize TF fragments (30-100 amino acids). Fragments are designed to have maximum immunogenicity and minimal overlap with other sequences in the human proteome. We have shown that scFvs isolated against purified TF fragments are able to identify full-length proteins in Western blot (WB), IP, and IP from formalin fixed chromatin fractions (FIG. 4). The latter property makes our scFvs potentially useful as a tool for ChIP studies.

As can be seen in FIG. 4A, purified ZNF384 fragment (amino acids 1-100) tagged with Maltose Binding Protein (MBP) tag and lysates of HEK293 cells transiently transfected with expression construct for full-length ZNF384 (DNASU) were separated on acrylamide gel, transferred to nitrocellulose membrane and probed with scFvs raised against ZNF384 fragment followed by anti-FLAG-HRP secondary Ab to detect scFvs (FLAG tagged). HEK293 cells transiently transfected with full-length ZNF622 and ZNF384 were lysed to prepare lysate (L) and chromatin (C) fractions (FIG. 4B). The lysates and chromatin fractions were incubated with scFvs (raised against ZNF384, amino acids 1-100, and ZNF622, amino acids 37-139) immobilized on FLAG beads. The beads were washed and the complexes were eluted with buffer containing 2% SDS and analyzed by Western Blot with anti-V5-HRP Ab to detect the presence of full-length TFs in eluates (tagged with V5 tag). FIG. 4C shows an experiment performed as in described above for FIG. 4B, except using formalin cross-linked cells following a chromatin immunoprecipitation (ChIP) protocol.

### Example 5

### Assembly of Reagents

Here, we describe the assembly of reagents, including the construction of a bi-functional library; selection of targets from the SGC and Interactome Data Sets; model systems; cloning of baits (corresponding to both peptides and full-length proteins) in Y2H and if deemed needed, M2H vectors; and construction and incorporation into a yeast vector of a positive selection system able to function in yeast.

### Hypothesis and Justification

We can develop a model system using, for example, peptides that we know have generated affinity reagents that were functional against full-length (FL) antigen (Ag). We can generalize this success onto an experimental set of up to 100-200 partial-Ags chosen from a set of new 25-50 full-length antigens (FL-Ag) from a group that includes transcription factors (TFs), kinases, and phosphatases. We can incorporate a yeast positive selectable marker into our pCH103-system to reduce or eliminate scFv clone interactors that do not bind to FL-Ag.

### Experimental Design and Expected Outcome

(a) Construction of scFv library in pCH103. If, for example, a new library is needed, a triplet-codon construction method can be used to construct a >10¹⁰ diversity library in pCH103 using triplet codon mutagenesis to specific sites within the CDRs (Mandrup et al., supra).

(b) Full-length antigen (FL-Ag) proteins. Two data sets will be used as a guide on the initial set of target FL-antigens:
(b1) Structural Genome Consortium (SGC) Data Set. The SGC has generated bacterial expression constructs for many kinases and phosphatases, and construct optimization has already been performed for a large group of them. A list of suitable Ags and partial Ags will be generated from a pre-selected set of proteins.
(b2) Interactome Data Set. The corresponding human interactome data set from one of our collaborators (M. Vidal) covering Space II and reported in 2014 [Rolland] is the largest experimentally-determined binary interaction map yet reported, with 13,944 interactions among 4,303 distinct proteins. We can use this data set of validated Y2H proteins as the starting point for antigen choice. Full-length clones will be purchased from DNASU (Arizona State University).

(c) Design of peptide targets. Criteria for choosing peptide and protein fragment antigens can include, for example: (i) surface-exposed hydrophilic turns, (ii) structural information (if known), (iii) uniqueness in the human proteome, (iv) absence of cysteine residues, (v) absence of known or predicted post-translational modification, (vi) removal of signal peptide, (vii) known or predicted splicing and polymorphic variation, and (viii) amino- and carboxyl-termini.

(d) Cloning into yeast. Homologous recombination is a well-established method for subcloning in yeast and can be used for our experiments.

(e) Positive selection in yeast. False positives have been a common problem with conventional Y2H. One class of false positives arises from induction of reporter gene transcription by baits or prey independent of their interaction. For example, a prey might possess intrinsic DNA binding activity and thus bring its fused transactivation domain to the promoter of the reporter gene. This type of false positive is quite rare in our system, arising from less than 0.01% of the prey proteins tested. This may be due, for example, to our use of three reporter genes (two selectable prototrophic markers, plus the β-Gal reporter). Increased stringency of interaction can be controlled using two selectable markers and the addition of 25 mM aminotriazole. We can also use the URA3 gene (orotidine-5′ -phosphate decarboxylase) as a counter-selectable marker. The endogenous URA3 gene can be inactivated, and a reporter cassette introduced, in which expression of URA3 is dependent on the presence of a bait/prey interaction. If URA3 is expressed, the uracil biosynthesis pathway will be functional. If 5-fluoroorotic acid (5-FOA) is added to the media, then the 5-FOA is metabolized into a suicide substrate for the essential thymidylate synthase enzyme. URA3/5-FOA systems are well known in the art and have been used successfully in yeast interaction trap systems to counter-select against bait/prey interactions in vivo (Vidal et al., PNAS 93(19):10315-20, 1996). The stringency of this counter-selection can be titrated by varying expression of the URA3 gene; higher levels of URA3 expression increase the sensitivity of a cell to 5-FOA.

### Alternate Options

Promiscuous interactors in φD and/or Y2H are dealt with in Example 6 below. Baits exhibiting either auto-activation or are shown not to translocate to the nucleus will not initially be chosen for further analysis using the proposed Y2H validation method. Baits not expressing in yeast as demonstrated by an absence of anti-V5 Ab binding to the V5 epitope will not initially be chosen for further analysis. In some cases, for example, TFs, it may be desirable to fuse the FL-Ag to the AD instead of the DBD. In such cases, we can construct a pCH103-analogous vector where the library is fused to the DBD instead of the AD. Or alternatively, we can use AXM cloning to transfer generated first-round hits into the alternative vector system. (5) The fusion to the carboxyl-end of the B42 AD may affect amino-derived fusions. If this occurs, we can separately test these rAbs in a vector system that reverses the order of fusion to the AD.

### Example 6

### In Vivo Assay to Validate Anti-Partial-Ags Against FL-Ag in Yeast

In this section, we describe an in vivo assay for validation of anti-partial antigens against full-length antigen in yeast. In brief, the assay involves: screen round 1 (or 2) enriched φD libraries against FL protein Ags in a round 2 (or 3) Y2H screen. Optionally, confirmation can be performed in a mammalian two-hybrid (M2H) system (Slentz-Kesler et al., Genomics 69(1):63-71, 2000).

### Hypothesis and Justification

Y2H can be used as a counter-screen between rounds of φD to enrich for affinity reagents able to bind to FL-proteins.

### Experimental Design and Expected Outcome

Several strategies will be used to alternate between φD and Y2H during the discovery rounds. In one example, the protocol incorporates a “ping-pong” screening involving alternating between φD in the initial rounds, followed by affinity-selection against partial-Ags in one or more rounds of φD (see, e.g., FIG. 1):
(a) φD enriched hits in pCH103 from (a) rounds 1 and/or round 2 will be transformed in bulk into a mating type a strain of yeast.
(b) FL-genes and partial-Ag gene fragments corresponding to FL-proteins and partial-Ags (listed in Specific Aim 1) will be cloned into the appropriate bait vectors and transformed into yeast mating type α.
(c) The enriched hits from step (b) will be mated to the baits in the yeast a strain from step (c). The mated yeast will be plated on the appropriate agar plates with selection.
(d) Individual yeast clones will be tested in ELISA against: (i) FL-Ag protein (if available), (ii) the original partial-Ag, (iii) one or more irrelevant FL-Ag proteins, and/or (iv) one or more irrelevant partial-Ags.
(e) Optionally, we can perform the yeast screen in bulk and then recover pooled clones against the FL-antigen in Y2H, followed by rescreening the clones though a round of φD against the partial-Ag.

### Alternate Options

Promiscuous interactors in φD will be eliminated by competition with an irrelevant peptide in solution during the affinity-selection steps. Promiscuous interactors during the Y2H screen can be eliminated by keeping the multi-wells separate during the yeast screen, counter-selection with FOA, and testing pools of the scFvs in the same wells in the Y2H screen against wells containing yeast expressing the hybrid fusion to: (i) the original partial-Ag, (ii) the FL-Ag protein, (iii) an irrelevant FL-Ag protein, and (iv) an irrelevant partial-Ag. scFvs from wells showing growth in either (iii) or (iv) can be eliminated from further analysis. (2) The method proposed may not work as well against membrane-bound and secreted proteins. We have developed a separate screening method for such antigens, involving an emulsion-based screen, as described, for example, in PCT Application No. PCT/US2013/076580 (incorporated herein by reference in its entirety). (3) Pooling and/or individual clone analysis may be needed to dissect hits from non-interactors and promiscuous interactors (see also Example 7).

### Example 7

### In Vitro Assays to Validate Affinity Reagents

Here, we perform several in vitro assays to validate the affinity reagents produced using the methods described above, including: immunoprecipitation (IP), enzyme-linked immunosorbent assay (ELISA), Western analysis, flow cytometry (FC), mass spectrometry (MS) and characterization of hits.

### Hypothesis and Justification

Affinity reagents will be tested in several applications, including, for example, ELISA and Western analyses against cell lysates of FLAG-tagged FL-Ag expressing cell lines.

### Experimental Design and Expected Outcome

(a) Optimization of selection rounds. We can vary the pooled-phage approach to additional rounds of stringent selection using the B2A-framework primary discovery library before Y2H selection.

(b) Measurement of affinity. To measure the affinity of scFvs and IgGs, we can rapidly evaluate selected hits from our library using a competitive AlphaScreen assay that we have developed, based on beads from PerkinElmer. Our pipeline typically yields rAbs with affinities in the single-digit nanomolar range, which is sufficient for most molecular biological and clinical applications. Additional rounds of affinity maturation can be used to further increase the affinity, if desired.

(c) Immunoprecipitation and mass spectrometry. IP has been described in the Materials and Methods (see, e.g., Example 1). Protein after polyacrylamide gel electrophoresis (PAGE) corresponding in size to the expected FL-Ag protein will be excised from Coomassie Blue stained polyacrylamide gel and submitted for MS analysis.

### Alternate Options

Certain human proteins may not be expressed well or in their proper confirmations, possibly due to the presence or absence of post-translational modification in a particular cell line. These antigens will preferably not be used for IP from cell lysates.

### Example 8

### Building a Scalable Approach for IgG Reformatting

Here, we describe methods for building a scalable approach for IgG reformatting. Exemplary validation assays that can be used include: IP, Western, ELISA, FACS, and cell-based assays.

### Hypothesis and Justification

(a) Although scFv or Fab systems facilitate the recombinant Ab selection process, the final desired format for leveraging the already available infrastructure for affinity reagents is generally a whole immunoglobulin (e.g., IgG). Lipopolysaccharide (LPS) contamination from bacterial protein production can interfere with in vitro cellular assays and in vivo functional screening. Hence, the present invention features the reformatting of scFv antibodies (e.g., scFvs generated and selected for desired binding properties according to the methods of the invention) directly into IgGs that can, for example, be expressed in mammalian cells.

(b) We are also developing specific antibody applications such as immunofluorescence (IF), ChIP, fluorescence activated cell sorting (FACS), and immunohistochemistry (IHC), which can be applied to some of the targets as required.

### Experimental Design and Expected Outcome

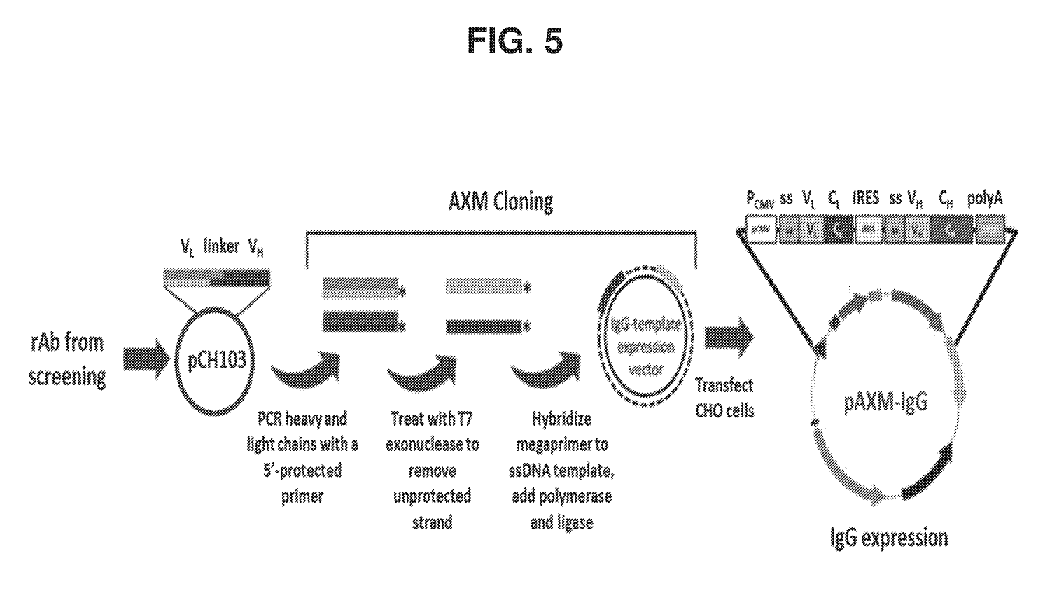

(a) scFv reformatting into IgGs. Heavy and light chain variable genes from IP-positive clones after affinity maturation for Ab production will be amplified and cloned into a single, dual-expression vector containing the constant regions of either mouse IgG2a or human IgG1 using a modified version of our AXM cloning method (FIG. 5). After addition of T7 polymerase and T4 ligase to extend the primers and ligate the product, the resulting heteroduplex will be transformed into E. coli strain AXE688, which expresses the Eco29kl endonuclease that restricts DNA containing Eco29kl sites. This approach will allow simultaneous cloning of the heavy and light chain variable genes in a single step. Since the template scFv will contain 6 Eco29kl sites within the heavy and light chain CDRs, only recombinant clones that have eliminated all 6 of these restriction sites will propagate in the AXE688 strain. We have shown that this method yields >95% recombinant clones (see, e.g., PCT Application No. PCT/US2014/068595, supra). Plasmid DNA will be isolated from the re-formatted clones.

(b) Antibody production. Antibody subcloning and production of IgGs has been described in the Materials and Methods section (see Example 1). The resulting IgGs will be purified in high-throughput using, for example, a protein A affinity resin.

### AXM Cloning

We have used the AXM cloning approach to generate hundreds of libraries for affinity maturation. The same principles are being applied in this proposal to convert the scFv rAbs to full IgGs. The vectors and cell lines we describe below have been used successfully in our laboratory to produce functional IgGs. As shown in FIG. 5, scFvs present in the pCH103 vector system will be reformatted into IgG genes in the pAXM-IgG vector. In brief, the variable heavy and light chain genes from the enriched scFvs will be amplified using reverse primers containing phosphorothioate linkages on the 5′ end. The resulting double-stranded DNA will be treated with T7 exonuclease to selectively degrade the unmodified strand of the dsDNA molecule. The resulting single-stranded DNA, or “megaprimer,” will then be annealed to a circular, single-stranded DNA containing the CMV promoter, IL2 signal sequences, internal ribosome entry site (IRES) and the light and heavy chain variable and constant domains with Eco29kl restriction sites in the CDR regions. The annealed megaprimers will be used to prime in vitro DNA synthesis by DNA polymerase. The ligated, heteroduplex product is then transformed into E. coli AXE688 cells, which express the Eco29kl restriction enzyme, and thus favor survival of the newly synthesized, recombinant strand that incorporates the megaprimer.

### Example 9

### Use of Yeast Two-Hybrid for Validation of scFvs Raised Against Peptides for Ability to Bind Full-Length Protein

The methods of the present invention may be used to determine if antibodies raised against peptides can be tested against full length protein, preferably without the need for actually purifying the full-length (FL) protein. Identifying and validating antibodies against FL proteins is often hindered by the availability of pure, properly folded immunogen. Peptides (e.g., peptide fragments of FL target proteins, such as target polypeptides and/or antigens as described herein) are often a readily available alternative target for raising antibodies (e.g., scFvs). In this example, the use of yeast two-hybrid (Y2H) technology for rapid validation of scFvs raised against peptides is described. In particular, such scFvs were evaluated for their ability to bind to FL target protein expressed and produced in the yeast cytoplasm. We found that scFvs can be expressed intracellularly in yeast and shown to interact with their targets.

### Overview of Two-Hybrid Assays

Two-hybrid assays that may be used in the present invention are known in the art. In one example, a transcription factor is used to determine if two proteins (referred to herein as the Bait and Prey proteins) interact. The transcription factor gene may include two domains, e.g., a binding domain and an activation domain (e.g., a LexA DNA binding domain and B42 activation domain, respectively). In some instances, the activation domain is essential for transcription of a reporter gene. The reporter may be, for example, an auxotrophic, colorimetric, or resistance gene. In this method, two fusion proteins are prepared: LexA-Bait and B42-Prey. Neither fusion protein alone is sufficient to initiate the transcription of the reporter gene. However, when both fusion proteins are produced, and the Bait portion of the first fusion protein interacts with the Prey portion of the second fusion protein, transcription of the reporter gene can proceed.

### Overview of Phage Display

Phage display is a laboratory technique well known in the art for the study of, e.g., protein-protein, protein-peptide, and protein-DNA interactions. An exemplary phage display assay uses bacteriophages to connect proteins with the genetic information that encodes them. In this example, a gene encoding a protein of interest is inserted into a phage coat protein gene, causing the phage to display the protein on its outside while containing the gene for the protein on its inside, thereby connecting the genotype and phenotype. These displaying phages can then be screened against target molecules (e.g., proteins, peptides, nucleic acids, and any other molecule of interest) to detect interaction between the displayed protein and one or more of the target molecules. In this way, large libraries of proteins can be screened and amplified. Exemplary bacteriophages that may be used in phage display include, without limitation, M13 and fd filamentous phage.

### Using Matchmaker Gold Yeast Two-Hybrid System (e.g., Clontech #630489)

In a first series of experiments, scFvs were raised against peptide fragments from a pair of target proteins (i.e., human Myb and human EZH2). The scFv-encoding genes were then cloned into vectors encoding a DNA binding domain (BD), thereby forming vectors encoding scFv-BD fusion proteins. Genes encoding portions of a target protein were cloned into vectors encoding an activation domain (AD), thereby forming vectors encoding target protein-AD fusion proteins. These vectors were then transformed singly into yeast strains. Haploid MATa yeast expressing scFv-BD fusion proteins were then mated to haploid MATα yeast expressing target protein-AD fusion proteins to form diploid yeast cells expressing both types of fusion proteins. If the scFv bound to the target protein in such cells, then the BD and AD would be brought into suitably close proximity to permit transcription factor activity and the expression of downstream reporter genes.

I. DNA Construction

Polynucleotides encoding the following scFvs were cloned into Ndel/Sall sites of the pGBKT7 BD cloning vector, as shown in Table 1:

TABLE 1
Fusing scFvs to Gal 4 DNA binding domain in pGBKT7 vector

| | | Full-Length | |
|---|---|---|---|
| scFv | | Target Transcrip- | UniProt |
| catalog # | Raised Against | tion Factor | ID |
| AXM1387 | Myb, aa 1-20, biotinylated | Human Myb | P10242 |
| | peptide | | |
| AXM1388 | Myb, aa 1-20, pSer11, | Human Myb | P10242 |
| | biotinylated peptide | | |
| AXM1389 | Myb, aa 455-544, SUMO | Human Myb | P10242 |
| | fusion, biotinylated | | |
| AXM2274 | EZH2, aa 342-441, MBP | Human EZH2 | Q15910 |
| | fusion, biotinylated | | |

Polynucleotides encoding the following protein fragments were cloned into Ndel/Xhol sites of the pGADT7 AD cloning vector:
1. Myb, aa 1-20 (pGADT7Myb20)
2. Myb, aa 455-544 (pGADT7Myb100)
3. EZH2, aa 342-441 (pGADT7EZH2100)
   All constructs were sequence confirmed, and midi-prep grade DNA was prepared for yeast transformation.

### II. Yeast Transformation

Y2H Gold strain (MATa, trp1-901, leu2-3, 112, ura3-52, his3-200, gal4Δ, gal80Δ, LYS2::GAL1 UAS-Gal1TATA-His3, GAL2UAS-Gal2TATA-Ade2 URA3::MEL1UAS-Mel1TATA AUR1-C MEL1) yeast, supplied with the Clontech system, were transformed with one of the following: (i) one of the pGBKT7-based constructs described above, (ii) empty pGBKT7 vector, or (iii) one of the control vectors, pGBKT7-p53 and pGBK-T7-Lam. Transformants were grown on SD/-Trp minimal agar plates.

Y187 strain (MATα, ura3-52, his3-200, ade2-101, trp1-901, leu2-3, 112, gal4Δ, gal80Δ, met−, URA3::GAL1UAS-Gal1TATA-LacZ, MEL1) yeast, supplied with the Clontech system, were transformed with one of the following: (i) one of the pGADT7-based constructs described above, (ii) empty pGADT7 vector, or (iii) the control vector, pGADT7-T (control, expected to show interaction with p53 but not with Lam). Transformants were grown on SD/-Leu minimal agar plates.

### III. Yeast Mating

One colony from each set of pGBKT7 and pGADT7 transformants was combined in 0.5 ml of YPDA media and incubated overnight at 30° C., shaking at 200 rpm. 0.1 ml of 1:10 dilution of the overnight mating culture was spread on plates made using one of the following minimal agar media:
DDO—double dropout (SD/-Leu/-Trp). All diploids would be expected to grow on this medium after mating
TDO—triple dropout (SD/-Leu/-Trp/-His). Growth is expected to occur if the two proteins interact. Less stringent. Weak binding will be detected
QDO—quadruple dropout (SD/-Leu/-Trp/-His/-Ade). Growth is expected to occur if the two proteins interact. More stringent. Strong binding will be detected.
DDO/X/A—double dropout (SD/-Leu/-Trp/X-α-Gal/Aurerobasidin A). Double dropout media supplemented with X-α-Gal, which detects α-Gal activity (secreted reporter from MEL1 gene in response to GAL4 activation), and Aurerobasidin A (yeast antibiotic, which is toxic to yeast unless they express mutant AUR1 gene). Most stringent. Colonies should grow blue if the two proteins interact.

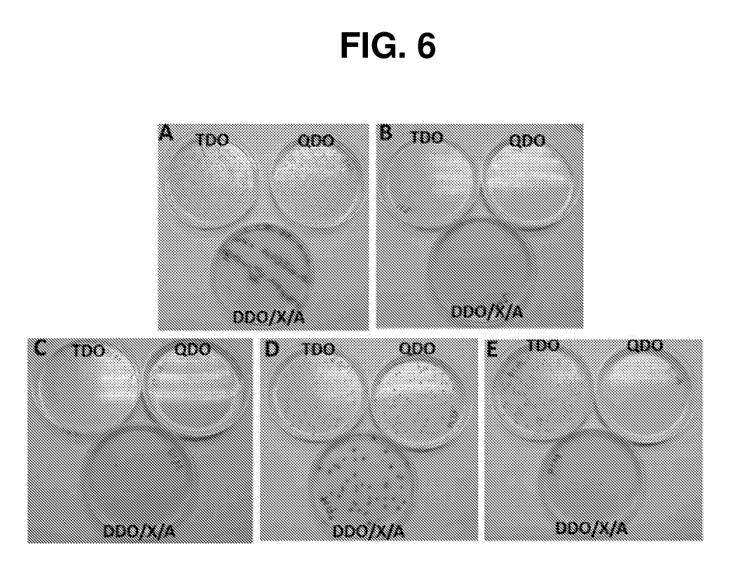

The results are described below in Table 2 and FIG. 6.

TABLE 2
Mating reactions

| | | | Mating | Interaction | Interaction | Weak or |
|---|---|---|---|---|---|---|
| Reaction # | MATa (Y2H Gold) | MATα (Y187) | Worked? | Expected? | Observed? | Strong? |
| 1 | pGBKT7-p53 | pGADT7-T | Y | Y | Y | Strong |
| 2 | pGBKT7-Lam | pGADT7-T | Y | N | N | |
| 3 | pGBKT7 empty | pGADT7 empty | Y | N | N | |
| 4 | pGBKT7 empty | pGADT7Myb20 | Y | N | N | |
| 5 | pGBKT7 empty | pGADT7Myb100 | Y | N | N | |
| 6 | pGBKT7 empty | pGADT7EZH2100 | Y | N | N | |
| 7 | pGBKT7-AXM1387 | pGADT7 empty | Y | N | N | |
| 8 | pGBKT7-AXM1387 | pGADT7Myb20 | Y | Y | Y | Weak |
| 9 | pGBKT7-AXM1387 | pGADT7Myb100 | Y | N | N | |
| 10 | pGBKT7-AXM1388 | pGADT7 empty | Y | N | N | |
| 11 | pGBKT7-AXM1388 | pGADT7Myb20 | Y | N | N | |
| 12 | pGBKT7-AXM1388 | pGADT7Myb100 | Y | N | N | |
| 13 | pGBKT7-AXM1389 | pGADT7 empty | Y | N | N | |
| 14 | pGBKT7-AXM1389 | pGADT7Myb20 | Y | N | N | |
| 15 | pGBKT7-AXM1389 | pGADT7Myb100 | Y | Y | Y | Strong |
| 16 | pGBKT7-AXM2274 | pGADT7 empty | Y | N | N | |
| 17 | pGBKT7-AXM2274 | pGADT7EZH2100 | Y | Y | Y | Weak |

### Using an Alternative Yeast Two-Hybrid System

In a second series of experiments, scFvs were raised against peptide fragments from a pair of target proteins (i.e., GRAP2, XIAP, and LNMA). The scFv-encoding genes were then cloned into vectors encoding an activation domain (AD), thereby forming vectors encoding scFv-AD fusion proteins. Genes encoding portions of a target protein were cloned into vectors encoding DNA binding domains (BD), thereby forming vectors encoding target protein-BD fusion proteins. These vectors were then transformed singly into yeast strains. Haploid MATa yeast expressing scFv-AD fusion proteins were then mated to haploid MATα yeast expressing target protein-BD fusion proteins to form diploid yeast cells expressing both types of fusion proteins. If the scFv bound to the target protein in such cells, then the BD and AD would be brought into suitably close proximity to permit transcription factor activity and the expression of downstream reporter genes.

I. Target Selection

Three proteins from the Vidal laboratory collection were selected as test cases: GRAP2, XIAP, and LNMA. These proteins have been shown to interact with their binding partners (LCP2, CASP9, and LMNB1, respectively) in multiple experimental conditions. As shown in Table 3 below, three peptides were designed and ordered for each protein, and then screened using our phage display scFv library to identify binders.

TABLE 3
Peptide design for the three protein targets

| | | | SEQ |
|---|---|---|---|
| Peptide | | Amino | ID |
| name | Sequence | acids | NO: |
| LMNA_1 | Biotin-GELHDLRGQVAKLEAALGEA | 160-179 | 2 |
| LMNA_2 | Biotin-RIDSLSAQLSQLQKQLAAKE | 298-317 | 3 |
| LMNA_3 | Biotin-DEYQELLDIKLALDMEIHAYRK | 357-378 | 4 |
| XIAP_1 | Biotin-SGSPVSASTLARAGFLYTGE | 38-57 | 5 |
| XIAP_2 | Biotin-THADYLLRTGQVVDISDTIY | 135-154 | 6 |
| XIAP_3 | Biotin-AEAVDKCPMCYTVITFKQK | 475-493 | 7 |
| GRAP2_1 | Biotin-GFFIIRASQSSPGDFSISVR | 78-97 | 8 |
| GRAP2_2 | Biotin-SLNKLVDYYRTNSISRQKQI | 124-143 | 9 |
| GRAP2_3 | Biotin-TDPVQLQAAGRVRWARALYD | 262-281 | 10 |

II. scFv Selection

The peptides shown in Table 3 were screened using AxioMx standard phage display methods to identify peptide-specific scFvs. Four unique scFvs generated against GRAP2 peptides GRAP2_2 and GRAP2_3 were selected, based on titration ELISA data, to move forward into Y2H testing.

III. DNA Construction

Anti-GRAP2 scFvs 1-4 were cloned into the pENTR23 vector using the BP Clonase kit (Life Technologies, 11789-013) and sequence confirmed using M13 Fw and Rev primers. The scFvs were fused to AD domain in the pDEST-AD vector using the LR Clonase kit (Life Technologies, 11791-019).

IV. Yeast Transformations

Y8800 strain (MATa, trp1-901, leu2-3, 112, ura3-52, his3-200, gal4Δ, gal80Δ, LYS2::GAL1-His3, GAL2-Ade2, Met2::Gal7-LacZ, cyh2^{R}) yeast were transformed with one of the following: (i) a vector encoding one of the four scFvs fused to the AD domain in the pDEST-AD vector, (ii) pDEST-AD-LCP2 (positive control interaction with GRAP2), or (iii) pDEST-AD CASP9 (negative control interaction with GRAP2). Transformants were grown on SD/-Trp minimal agar plates.

Y8039 strain (MATα, trp1-901, leu2-3, 112, ura3-52, his3-200, gal4Δ, gal80Δ, LYS2::GAL1-His3, GAL2-Ade2, Met2::Gal7-LacZ, cyh2^{R}) yeast were transformed with pDEST-DB-GRAP2. Transformants were grown on SD/-Leu minimal agar plates. The following controls were also used: pGBKT7-P53/pGADT7-T and pGBKT7-AXM1389/pGADT7-Myb100.

V. Yeast Mating

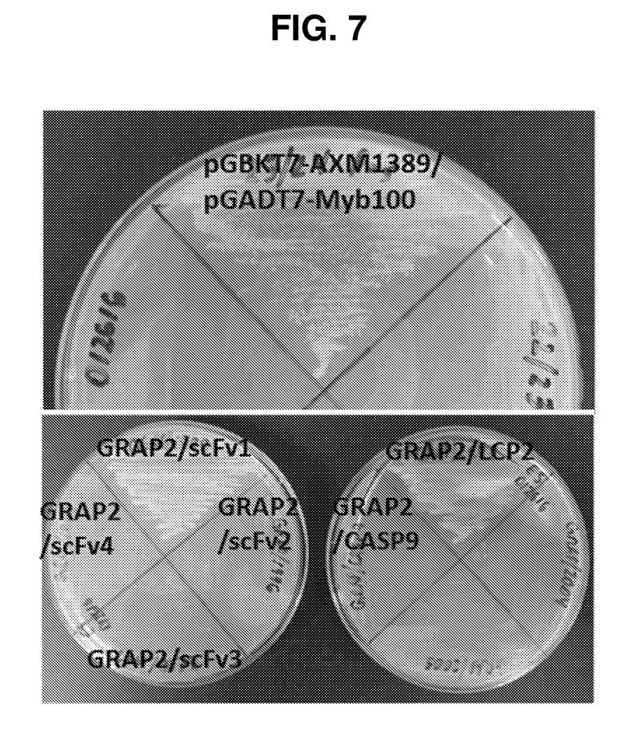

Yeast mating was performed as described above. The mating reactions were spread on SD/-Trp/-Leu plates as well as SD/-Trp/-Leu/-His+1 mM 3AT plates to detect interactors. One of the four scFvs selected against GRAP2 peptide, AXM1389, showed interaction with full-length GRAP2 (FIG. 7).

### Other Embodiments

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of identifying and validating a binding moiety as capable of binding to a target polypeptide, said method comprising:
(a) providing a plurality of viruses, each virus comprising:
a nucleic acid encoding a binding moiety, wherein said binding moiety is displayed on the surface of the virus;
(b) incubating said plurality of said viruses with said target polypeptide or a peptide fragment thereof;
(c) examining whether said binding moieties displayed by said viruses bind to said target polypeptide, or to said peptide fragment thereof;
(d) expressing, for each of said binding moieties identified as capable of binding to said target polypeptide, in a cell:
(i) a first fusion protein comprising a particular binding moiety identified as capable of binding to said target polypeptide and a first reporter moiety, and
(ii) a second fusion protein comprising said target polypeptide, or a peptide fragment thereof, and a second reporter moiety,
wherein binding of said particular binding moiety to said target polypeptide, or to said peptide fragment thereof, results in expression in said cell of a detectable gene, wherein expression of said detectable gene is under the control of said first reporter moiety and said second reporter moiety; and
(e) determining if said detectable gene is expressed by each of said cells, thereby validating said binding moieties identified as capable of binding to said target polypeptide as capable of binding to said target polypeptide.

2. The method of claim 1, wherein a plurality of binding moieties are identified in said examining step and said sequencing step as capable of binding to said target polypeptide, said method further comprising expressing each of said identified binding moieties in a distinct cell according to step (d) and determining if said detectable gene is expressed by each of said distinct cells according to step (e).

3. The method of claim 1 or 2, further comprising generating a plurality of variants of at least one of said validated binding moieties and repeating steps (a)-(e) using said plurality of variants as said binding moieties of step (a).

4. A method of validating a binding interaction between a binding moiety and a target polypeptide, said method comprising:
expressing, in a cell:
(a) a first fusion protein comprising a binding moiety and a first reporter moiety, and
(b) a second fusion protein comprising a target polypeptide, or a peptide fragment thereof, and a second reporter moiety;
said binding moiety having been identified as capable of binding to said target polypeptide by:
(i) expressing a nucleic acid encoding said binding moiety on a virus, wherein said binding moiety is displayed on the surface of the virus,
(ii) incubating said virus with said target polypeptide, or said peptide fragment thereof, and
(iii) examining whether said binding moiety displayed by said virus binds to said target polypeptide, or to said peptide fragment thereof;
wherein binding of said binding moiety to said target polypeptide, or to said peptide fragment thereof, in said cell results in said cell expressing a detectable gene, wherein expression of said detectable gene is under the control of said first reporter moiety and said second reporter moiety; and
determining if said detectable gene is expressed by said cell, thereby validating said binding moiety as capable of binding to said target polypeptide.

5. The method of claim 4, further comprising repeating said expressing step and said determining step with one or more additional binding moieties having been identified as capable of binding to said target polypeptide according to steps (i)-(iii).

6. The method of any one of claims 1-5, wherein said binding moiety is an antibody or antibody fragment.

7. The method of claim 6, wherein said binding moiety is a single-chain variable fragment (scFv).

8. The method of claim 7, wherein said scFv comprises an antibody framework comprising an amino acid sequence sharing at least 90% sequence identity with SEQ ID NO: 1.

9. The method of any one of claims 1-8, wherein said incubation step comprises incubating said virus with a peptide fragment of said target polypeptide.

10. The method of claim 9, wherein said peptide fragment of said target polypeptide is less than about 40 amino acids in length.

11. The method of claim 9, wherein said peptide fragment of said target polypeptide is greater than about 40 amino acids in length.

12. The method of claim 9, wherein said peptide fragment of said target polypeptide is about 30-100 amino acids in length.

13. The method of any one of claims 9-12, wherein said peptide fragment is synthetic.

14. The method of any one of claims 1-13, wherein said second fusion protein comprises the full length amino acid sequence of said target polypeptide.

15. The method of any one of claims 1-14, wherein said target polypeptide is post-translationally modified.

16. The method of claim 15, wherein said target polypeptide is phosphorylated.

17. The method any one of claims 1-16, wherein said target polypeptide is a soluble protein.

18. The method of claim 17, wherein said target polypeptide is an intracellular protein.

19. The method of any one of claims 1-18, wherein said first reporter moiety is a transcription factor activation domain and said second reporter moiety is a DNA binding domain.

20. The method of any one of claims 1-18, wherein said first reporter moiety is a DNA binding domain and said second reporter moiety is a transcription factor activation domain.

21. The method of any one of claims 1-20, wherein said cell is a yeast cell, mammalian cell, bacterial cell, insect cell, or plant cell.

22. The method of claim 21, wherein said yeast cell is Saccharomyces cerevisiae or Schizosaccharomyces pombe.

23. The method of any one of claims 1-22, wherein said virus is bacteriophage M13.

24. The method of any one of claims 1-23, wherein said examining step comprises performing an enzyme-linked immunosorbent assay (ELISA), immunoprecipitation, Western blot, flow cytometry, or mass spectrometry.

25. The method of any one of claims 1-24, wherein each of said viruses originates from a bi-functional vector comprising a gene encoding a particular binding moiety, wherein:
if said bi-functional vector is present in a first cell, said bi-functional vector acts as a template for expression by said first cell of a first fusion protein comprising said particular binding moiety and a viral protein; and
if said bi-functional vector is present in a second cell, said bi-functional vector acts as a template for expression by said second cell of a second fusion protein comprising said particular binding moiety and a reporter moiety.

26. The method of claim 25, wherein said first cell is a bacterial cell.

27. The method of claim 25 or 26, wherein said second cell is a yeast cell and said reporter moiety is a transcription factor activation domain or a DNA binding domain.

28. The method of claim 27, wherein said transcription factor activation domain is a B42 domain.

29. The method of any one of claims 25-28, wherein said bi-functional vector comprises a suppressible stop codon located between said viral protein and said binding moiety.

30. The method of claim 29, wherein said suppressible stop codon is an amber stop codon.

31. The method of any one of claims 25-30, wherein said viral protein is a gp3 protein.

32. The method of any one of claims 25-31, wherein said bi-functional vector is a pCH103 vector.

33. The method of any one of claims 1-32, wherein said expressing step and said determining step are performed in liquid media.

34. The method of any one of claims 1-33, wherein said cell lacks a selectable marker prior to said expressing step, and said expressing step further comprises expressing said selectable marker in said cell.

35. The method of claim 34, wherein said selectable marker is URA3.

36. The method of any one of claims 1-35, wherein said nucleic acids encoding said binding moieties in said plurality of viruses are generated by:
(i) providing a template DNA molecule comprising a binding moiety sequence,
(ii) providing a pair of oligonucleotides, wherein said oligonucleotides hybridize to opposite strands of said binding moiety sequence, wherein one of said oligonucleotides is protected, the other oligonucleotide is non-protected, and said oligonucleotides flank said binding moiety sequence;
(iii) performing an amplification reaction on said template DNA molecule using said oligonucleotides, thereby generating a population of dsDNA variants of said binding moiety sequence;
(iv) incubating said population of dsDNA variants with an enzyme capable of selectively degrading the non-protected strand over the protected strand of said dsDNA variants, thereby producing a population of ssDNA variants of said binding moiety sequence;
(v) hybridizing said population of ssDNA variants to ssDNA intermediaries, wherein said ssDNA intermediaries comprise a sequence substantially identical to said binding moiety sequence or a fragment thereof, generating heteroduplex DNA; and
(vi) transforming said heteroduplex DNA into host cells, thereby generating a plurality of variants of said binding moiety sequence.

37. The method of claim 36, wherein said template DNA molecule further comprises viral nucleic acid sequences.

38. The method of claim 36 or 37, further comprising cloning said variants of said binding moiety sequence into a viral vector.

39. The method of any one of claims 36-38, wherein said nonrecombinant copies of said binding moiety sequence comprise a predetermined restriction site, and recombinant copies of said binding moiety sequence do not comprise said predetermined restriction site.

40. The method of claim 39, wherein said host cells express a restriction enzyme that recognizes and cleaves said predetermined restriction site.

41. The method of claim 40, wherein said transformation step further comprises incubating said host cells under conditions in which said restriction enzyme can cleave nucleic acids having said predetermined restriction site.

42. The method of claim 40 or 41, wherein said restriction enzyme is Eco29kl.

43. The method of any one of claims 36-42, wherein said host cells are bacteria.

44. The method of claim 43, wherein said host cells are AXE688 E. coli.

45. The method of any one of claims 36-44, wherein said template DNA molecule is a viral vector.

46. The method of any one of claims 36-45, wherein said template DNA molecule is a bi-functional vector, wherein:
if said bi-functional vector is present in a first cell, said bi-functional vector acts as a template for expression by said first cell of a first fusion protein comprising said binding moiety sequence and a viral protein; and
if said bi-functional vector is present in a second cell, said bi-functional vector acts as a template for expression by said second cell of a second fusion protein comprising said binding moiety sequence and a reporter moiety.

47. The method of claim 46, wherein said bi-functional vector is a pCH103 vector.

48. The method of any one of claims 36-47, wherein said enzyme capable of selectively degrading the non-protected strand over the protected strand of said dsDNA variants is a T7 exonuclease.

49. The method of any one of claims 1-48, further comprising, after said determining step, immunoprecipitating said target polypeptide from a transiently-transfected cell.

50. The method of claim 49, wherein said target polypeptide to be immunoprecipitated is tagged with an epitope tag.

51. The method of claim 50, wherein said epitope tag is FLAG, HA, Myc, His, V5, GFP, YFP, GST, or MBP.

52. The method of any one of claims 49-51, wherein said transiently-transfected cell is a mammalian cell.

53. An antibody framework comprising an amino acid sequence sharing at least 90% sequence identity with SEQ ID NO: 1.

54. A nucleic acid encoding the antibody framework of claim 53.
